(19) United States
(12) Patent Application Publication
(10) Pub. No.: US 2003/0022318 A1
(43) Pub. Date: Jan. 30, 2003

(54) Method for thermocycling amplification of nucleic acid sequences and the generation of related peptides thereof

(75) Inventors: Shi-Lung Lin, Alhambra, CA (US); Shao-Yao Ying, San Marino, CA (US)

Correspondence Address: LYON & LYON LLP, 633 WEST FIFTH STREET, SUITE 4700, LOS ANGELES, CA, 90071, US

(73) Assignee: EPICLONE, INC.

(21) Appl. No.: 09/949,305

(22) Filed: Sep. 7, 2001

Related U.S. Application Data
(63) Continuation-in-part of application No. 09/494,212, filed on Jan. 25, 2000.

(51) Int. Cl. C12P 19/34; C12Q 1/68

(52) U.S. Cl. 435/91.2; 435/6

(57) ABSTRACT

The present invention provides a fast, simple and reliable polymerase thermocycling reaction procedure for the linear amplification of nucleic acid sequences from cellular RNAs or genomes or both. The principle of this polymerase thermocycling reaction method relies upon the thermal cycling steps of promoter-linked nucleic acid template synthesis and in-vitro transcriptional amplification to bring up the amount of desired nucleic acid sequences up to two thousand fold within one cycle of the above procedure. Neither RNase H activity nor alkaline degradation is used in order to protect the RNA sequences of resulting amplified products which are ready for further applications, such as genechip/microarray analysis, in-vitro translation reaction and induction of RNA interference. Without the preferential amplification drawback of PCR/RT-PCR methods, the accuracy and resolution of current molecular biology analysis and diagnosis can be significantly improved by the present invention.

This application is a continuation-in-part application that claims priority to co-pending U.S. patent application Ser. No. 09/494,212, filed Jan. 25, 2000, which is hereby incorporated by reference as if fully set forth herein.

### BACKGROUND OF THE INVENTION

1. Field of the Invention

The present invention generally relates to the field of methods for generating nucleic acid sequences with an enzymatic thermocycling procedure. More particularly, the present invention relates to the field of polymerase thermocycling reaction methods of interchangeably amplifying both RNA and DNA sequences from cellular RNAs and/or genomes.

2. Description of the Prior Art

The following references are pertinent to this invention:

1. Sambrook et. al., “Molecular Cloning, 2nd Edition”, Cold Spring Harbor Laboratory Press, pp. 8.11-8.19 (1989).

2. Shi-Lung Lin, Cheng-Ming Chuong and Shao-Yao Ying; “A novel mRNA-cDNA interference phenomenon for silencing bc1-2 expression in human LNCaP cells”, Biochem. Biophys. Res. Commun. 281: 639-644 (2001).

3. Van Gelder et.al., “Amplified RNA synthesized from limited quantities of heterogeneous cDNA”, Proc. Natl. Acad. Sci. USA 87: 1663-1667 (1990).

4. O'Dell et.al., “Amplification of mRNAs from single, fixed, TUNEL-positive cells”, BioTechniques 25: 566-570 (1998).

5. Eberwine et.al. , “Analysis of gene expression in single live neurons”, Proc. Natl. Acad. Sci. USA 89: 3010-3014 (1992).

6. Compton, J., “Nucleic acid sequence-based amplification”, Nature 350: 91-92 (1991).

7. Shi-Lung Lin, Cheng-Ming Chuong, Randall B. Widelitz and Shao-Yao Ying; “In vivo analysis of cancerous gene expression by RNA-polymerase chain reaction”, Nucleic Acid Res. 27: 4585-4589 (1999).

8. U.S. Pat. No. 4,683,202 issued to Mullis et.al.

9. U.S. Pat. No. 4,965,188 issued to Mullis et.al.

10. U.S. Pat. No. 5,817,465 issued to Mallet et.al.

11. U.S. Pat. No.5,514,545 issued to Eberwine et.al.

12. U.S. Pat. No. 6,197,554 issued to Lin et.al.

13. U.S. Pat. No. 5,888,779 issued to Kacian et.al.

14. Shi-Lung Lin, Cheng-Ming Chuong and Shao-Yao Ying; “Microarray profiling of gene expression at the single cell scale”, Science, submitted (2001).

The ability to amplify nucleic acid sequences from cells has permitted the molecular investigations of intracellular gene and/or genome status under certain special conditions, such as pathogenesis, mutation, treatment processing and developmental control. Traditionally, nucleic acid sequences are isolated from genomic DNAs and/or cellular RNAs (Sambrook et.al., “Molecular Cloning, 2nd Edition”, pp. 8.11-8.35 (1989)). However, the tedious procedures of extraction, purification and cloning usually fail to maintain the completeness of all nucleic acid sequences, and result in a significant loss of rare DNA/RNA populations. The requirement of bulk tissue samples for nucleic acid extraction is another drawback for previous methods. Current genetic/genomic analysis and molecular diagnosis all rely on relatively pure sample collections with high throughput and high resolution capacity. Unfortunately, it is impossible to collect enough pure/homogeneous samples for the traditional extraction methods due to the fast degradation rate of nucleic acid sequences, especially messenger RNAs (mRNA).

On the other hand, the generation of amplified DNA products by polymerase chain reaction (PCR) and/or reverse transcription (RT-PCR) has become the most common way among current nucleic acid amplification methods. Prior art attempts at amplifying DNA sequences with PCR, such as U.S. Pat. Nos. 4,683,202 and 4,965,188 to Mullis, and with RT-PCR, such as U.S. Pat. No. 5,817,465 to Mallet, uses DNA polymerases and/or reverse transcriptase to generate DNA products based on a thermal cycling strategy. Although the PCR and RT-PCR methods successfully produce high quantity of double-stranded DNAs from either a DNA or RNA template, the low fidelity of their DNA products is usually a noted problem which results from the high mis-reading rate of most thermostable DNA polymerase activities after multiple thermocycling processes. Moreover, the resulting products of PCR and RT-PCR are double-stranded DNAs which cannot be used in genechip/microarray analysis, in-vitro translation reaction and RNA interference assays (Lin et.al., Biochem. Biophys. Res. Commun. 281: 639-644 (2001)). Furthermore, the preferential amplification of some nonspecific products occurs very often in a PCR/RT-PCR reaction, incurring inevitable bias and difficulty in genetic/genomic analysis (Sambrook et.al., “Molecular Cloning, 2nd Edition”, pp. 8.11-8.35 (1989)). These disadvantages diminish the accuracy of PCR/RT-PCR analysis in both genetic research and clinical diagnosis.

The generation of antisense RNA sequences (aRNA) with in-vitro transcription reaction has provided linear amplification of nucleic acid sequences from limited cells (Van Gelder et.al., Proc. Natl. Acad. Sci. USA 87: 1663-1667 (1990)). Prior art attempts at aRNA amplification, such as U.S. Pat. No. 5,514,545 to Eberwine and U.S. patent application No. 6,197,554 to Lin., use reverse transcription to incorporate an RNA promoter into a DNA template for further transcriptional amplification of aRNA (Eberwine et. al., Proc. Natl. Acad. Sci. USA 89: 3010-3014 (1992)). Although these aRNA amplification methods lead to the identification of some useful mRNA markers for disease detection, the rare mRNA-representative copies are not amplified due to the low affinity of oligo(dT)-promoter primers which are widely used in the current aRNA-related methods (O'Dell et. al., BioTechniques (1998)). Based on the principle of aRNA amplification, the generation of genomic nucleic acid sequences is obviously not available.

Alternatively, an improved RNA amplification method, nucleic acid sequence-based amplification (NASBA; Compton, Nature 350: 91-92 (1991)), has used another kind of promoter-linked primers with sequence-specific affinity for similar kinds of transcriptional amplification as the aforementioned aRNA methods. Prior art attempts at NASBA, such as U.S. Pat. No. 5,888,779 to Kacian, can linearly amplify double-stranded DNAs at very stable temperature conditions, but not the thermocycling procedure because of enzyme sensitivity. The RNA promoter is introduced into DNA templates by both reverse transcriptase and ribonuclease H (RNase H) activities and/or strong alkaline conditions. Therefore, the resulting products lack RNA portions due to the use of RNase H and/or alkaline chemicals that degrade most of the RNA products. Although this method successfully provides a linear amplification of DNA products from both mRNAs and genomic DNAs, the generation of highly pure single-stranded RNA products which are needed for genechip/microarray analysis and in-vitro translation is still not accessible. An improved thermocycling procedure has been proposed to potentially overcome part of this problem (Lin et.al., Nucleic Acid Res. 27: 4585-4589 (1999)).

In summary, it is desirable to have a fast, simple and reliable thermocycling method for generating amplified nucleic acid sequences without using degradation agents thereof, such as ribonuclease activities and alkaline conditions. The amplified products may be applied to screen differential gene sequences, to search for functional domains in genes and/or genomes, to produce synthetic peptides in vitro, and to design diagnoses and/or therapies for diseases.

### SUMMARY OF THE INVENTION

The present invention is a novel polymerase chain reaction method which amplifies messenger RNAs from single cells.

Described in detail, a preferred embodiment of the present invention method includes the following steps:

a. preventing a plurality of messenger RNAs from degradation, wherein said messenger RNAs are protected to be intact along with following steps;

b. contacting said messenger RNAs with a plurality of oligodeoxythymidylate nucleotide sequences to form a plurality of first-strand complementary DNAs, wherein said first-strand complementary DNAs are generated by reverse transcription of said messenger RNAs with said oligodeoxythymidylate nucleotide sequences as primers;

c. permitting 3′-end extension of said first-strand complementary DNAs to form a plurality of polynucleotide-tailed first-strand complementary DNAs, wherein said polynucleotide-tailed first-strand complementary DNAs are extended by terminal transferase activity with multiple copies of same deoxynucleotides to form polynucleotide tails;

d. incubating denatured said polynucleotide-tailed first-strand complementary DNAs with a plurality of oligo(antisense polynucleotide)-promoter primers to form a plurality of double-stranded complementary DNAs, wherein said double-stranded complementary DNAs are generated by extension of DNA polymerase activity with said oligo(antisense polynucleotide)-promoter primers complementary to the polynucleotide tails of said polynucleotide-tailed first-strand complementary DNAs;

e. permitting transcription of said double-stranded complementary DNAs to form a plurality of amplified messenger RNAs, wherein said amplified messenger RNAs are generated by extension of RNA polymerase activity through the promoter region of said double-stranded complementary DNAs; and

f. contacting said amplified messenger RNAs with said oligodeoxythymidylate nucleotide sequences to form a plurality of said polynucleotide-tailed first-strand complementary DNAs, wherein said polynucleotide-tailed first-strand complementary DNAs are generated by reverse transcription of said amplified messenger RNAs with said oligodeoxythymidylate nucleotide sequences as primer.

In one aspect of this embodiment, the cycling steps of (d) through (f) can be repeated at least one time for the amplification of said messenger RNAs. According to another aspect of this preferred embodiment, the final nucleotide products are preserved in the form of double-stranded duplexes to prevent the degradation of amplified messenger RNAs, preferably, in the form of RNA-DNA hybrid duplexes in the step (f). The formation of the RNA-DNA hybrid duplexes is preferably accomplished by the activities of reverse transcriptases, such as AMV, M-MuLV and HIV-1 reverse transcriptases at a temperature ranged from about 37° C. to about 52° C., and/or Tth-like polymerases with reverse transcription activity at a temperature ranged from about 55° C. to about 72° C., such as thermostable Tth and C. therm. polymerases. The Tth-like polymerase refers to a both RNA- and DNA-directed DNA polymerase which can perform DNA polymerization from both RNA and DNA templates.

The mRNAs can be prepared from a plurality of fixed cells, wherein said fixed cells are protected from RNA degradation and also subjected to permeabilisation for enzyme penetration. Those fixed cells include fixative-treated cultural cells, frozen fresh tissues, fixative-treated fresh tissues or paraffin-embedded tissues on slides. To increase the transcriptional production of mRNAs in the step (e), the promoter sequences are preferably incorporated into the 5′-ends of said second-strand cDNAs. In another aspect of this embodiment, said amplified mRNAs are preferably capped by P¹-5′-(7-methyl)-guanosine-P³-5′-adenosine-triphosphate or P¹-5′-(7-methyl)-guanosine-P³-5′-guanosine-triphosphate in the step (e) for further in vitro translation. On the other hand, the deoxynucleotide used in the tailing reaction of said first-strand complementary DNAs is either deoxyguanylate (dG) or deoxycytidylate (dC), and the average number of tailed nucleotides is larger than seven; most preferably, the number is about twelve. Advantageously, the final amplified mRNAs can be continuously reverse-transcribed into double-stranded cDNA by Tth-like DNA polymerase activity, such as thermostable Tth and C. therm. polymerases. The final double-stranded cDNAs are preferably cloned into competent vectors for further applications, such as transfection assay, differential screening, functional detection and so on. The formation of the double-stranded cDNAs is preferably accomplished by the activities consisting of E. coli DNA polymerase 1, Klenow fragment of E. coli DNA polymerase 1 and T4 DNA polymerase at about 37° C., and/or Taq DNA polymerases, Pwo DNA polymerases, Pfu DNA polymerases and Tth-like thermostable polymerases at about 70° C.

Alternatively, the present invention is an improved polymerase thermocycling reaction method which amplifies nucleic acid sequences from either cellular RNAs or genomes or both.

Described in detail, a preferred embodiment of the present invention method includes the following steps:

a) permitting the denaturation of a plurality of nucleic acid templates for the step b) hereafter;

b) obtaining a starting solution by adding to a buffered condition comprising said denatured nucleic acid templates, a primer, a promoter-containing primer, a plurality of deoxynucleotide triphosphates, a plurality of ribonucleotide triphosphates, a sufficient amount of enzyme activities containing reverse transcription, DNA-dependent DNA polymerase and RNA polymerase activities, wherein said buffered condition is sufficient to maintain said enzyme activities in the steps hereafter;

c) contacting said promoter-containing primer with said nucleic acid templates at a predetermined temperature sufficient to form stable annealing interactions, and maintaining said predetermined temperature for sufficient time, whereby a plurality of promoter-containing nucleic acid templates are generated;

d) heating said promoter-containing nucleic acid templates to a temperature sufficient to permit denaturation, and maintaining said temperature for a sufficient time to provide denaturation of said promoter-containing nucleic acid templates without inactivating said enzyme activities containing reverse transcription and DNA-dependent DNA polymerase activities;

e) contacting said primer with said denatured promoter-containing nucleic acid templates at a predetermined temperature sufficient to form stable annealing interactions, and maintaining said predetermined temperature for sufficient time whereby a plurality of promoter-containing double-stranded DNA templates are generated with a desired size sufficient to permit performance of step f) hereafter, wherein said desired size is a plurality of fragment sequences of said nucleic acid templates flanked with said promoter-containing primer in one end and said primer in the other end of the other orientation;

f) permitting transcriptional amplification of said promoter-containing double-stranded DNA templates at a predetermined temperature sufficient to form a plurality of amplified RNA sequences, wherein said amplified RNA sequences are generated by said enzyme activities containing RNA polymerase activities through the promoter region of said promoter-containing double-stranded DNA templates;

g) contacting said amplified RNA sequences with said primer at a predetermined temperature sufficient to form stable annealing interactions, and maintaining said predetermined temperature for sufficient time, whereby a plurality of complementary DNAs are synthesized and a plurality of DNA-RNA hybrid templates are formed; and

h) heating said DNA-RNA hybrid templates to a temperature sufficient to permit denaturation, and maintaining said temperature for a sufficient time to provide denaturation of said DNA-RNA hybrid templates without inactivating said enzyme activities containing reverse transcription and DNA-dependent DNA polymerase activities; so as to provide said denatured nucleic acid templates for the step b) herebefore and hereafter.

Alternatively defined in detail, the present invention is a kit for an improved polymerase thermocycling reaction procedure which provides linear amplification of nucleic acid sequences from either cellular RNAs or genomes or both, comprising the components of:

a) a plurality of nucleic acid templates;

b) a plurality of conditioned buffers;

c) a plurality of primers;

d) a plurality of promoter-containing primers;

e) a plurality of deoxynucleotide triphosphates;

f) a plurality of ribonucleotide triphosphates; and

g) a sufficient amount of enzyme activities containing reverse transcription, DNA-dependent DNA polymerase and RNA polymerase activities.

In one aspect of this embodiment, the thermocycling steps of (b) through (h) can be repeated at least one time for the linear amplification of said nucleic acid templates from either cellular RNAs or genomic DNAs or both. Advantageously, the cycling reactions of step (b) to (h) can be continuously performed in a united buffer containing Tris-HCl, about pH 8.3 at 25° C., KCl/NaCl, MgCl₂, dithiothreitol and/or betaine, such as one fold of RT&T buffer (40 mM Tris-HCl, pH 8.3 at 25° C., 30 mM KCl, 8 mM MgCl₂, and 10 mM DTE). This low stringent buffer condition also facilitates the annealing interaction between a promoter-containing primer and its nucleic acid templates at certain predetermined temperatures. Also, the use of thermostable enzymes advantageously improve the simplicity, stability and efficiency of this thermocycling procedure, such as Tth-like thermostable polymerases which refer to RNA- and DNA-dependent DNA polymerases with reverse transcription activity. Because the capability of using transcriptional amplification of RNA and DNA sequences (FIG. 5), our invention advantageously provides more flexibility in the enzymatic synthesis of single-stranded RNAs, RNA-DNA hybrids and double-stranded DNAs which are ready for a variety of biochemical applications such as genomic and cDNA library preparation, probe preparation, in-vitro translation and gene knockout analysis (RNA interference).

The nucleic acid sequences can be prepared from a plurality of fixed cells, wherein said fixed cells are protected from RNase/DNase degradation by heat, chemicals and/or RNase inhibitors. Those fixed cells are also subjected to permeabilisation for better enzyme penetration, including fixative-treated cultural cells, frozen fresh tissues, fixative-treated fresh tissues or paraffin-embedded tissues on slides. For transcriptional amplification in the step (f), a promoter-containing primer can be incorporated into the 5′-regions of said nucleic acid templates in either orientation. Such transcriptional amplification is preferably accomplished by the activities of T3, T7, SP6 and/or M13 RNA polymerases at about 37° C. During the transcriptional amplification reaction, said amplified RNA sequences are preferably capped by P¹-5′-(7-methyl)-guanosine-P³-5′-adenosine-triphosphate or P¹-5′-(7-methyl)-guanosine-P³-5′-guanosine-triphosphate in the step (f) for further in vitro translation. Advantageously, said amplified RNA sequences can be continuously transformed into promoter-containing double-stranded DNAs preferably by a Tth-like thermostable polymerase with reverse transcription activity. The labeling of resulting products can be achieved by the incorporation of labeled deoxynucleotides during the step (g) and/or labeled ribonucleotides during the step (f).

In a further embodiment, is provided a method for the differential screening of tissue specific gene expression at a cellular level. This method includes generating a gene expression profile of cells that are dissected from a homogeneous cell region of a tissue sample by a single-cell isolation device such as a tissue-shredder columns, micromanipulators or laser-capture devices. This method involves comparing the gene expression profile of the cells to another gene expression profile by microarray and/or subtractive hybridizations and selecting the differently expressed genes from the compared gene expression profiles; so as to provide a differentially expressed gene profile of the tissue cells.

Furthermore, a method for preparing labeled RNA/DNA probes for a gene chip technology, is also provided. This method includes the generation of a mRNA library of cells of interest. It involves combining the mRNA library with a primer, a promoter-containing primer, a kind of labeled deoxynucleotide or ribonucleotide triphosphates (i.e., labeled by a chemical such as biotin, avidin, digoxigenin fluorescein, Cy3, Cy5 and radioactive isotopes), a plurality of unlabeled deoxynucleotide and ribonucleotide triphosphates, a reverse transcription enzyme, a DNA-dependent DNA polymerase enzyme and an RNA polymerase enzyme. A further step includes contacting the promoter-containing primer with the RNA templates of the mRNA library to generate a plurality of promoter-containing nucleic acid templates. The promoter-containing nucleic acid templates are then denatured and contacted with the primer to generate a plurality of promoter-containing double-stranded DNA templates, wherein the double-stranded nucleic acid templates are flanked with said promoter-containing primer in one end and said primer in the other end of the other orientation. Further steps include, transcribing the promoter-containing double-stranded DNA templates to form a plurality of labeled RNA probes; reverse-transcribing the RNA probes to form a plurality of labeled DNA probes; and contacting the labeled RNA or DNA probes with a gene chip or microarray; so as to provide an expression profile of the interested cells.

The present invention also includes a method for cloning full-length sequences of unknown gene transcripts, which includes denaturing a plurality of nucleic acid templates containing a gene or its transcript which is desired to be cloned, combining the denatured nucleic acid templates with a primer complementary to the 3′-end of said desired gene, a promoter-containing primer homologous to the 5′-end of said desired gene, a plurality of deoxynucleotide triphosphates, a plurality of ribonucleotide triphosphates, a reverse transcription enzyme, a DNA-dependent DNA polymerase enzyme and an RNA polymerase enzyme and contacting the primer with the nucleic acid templates to generate a plurality of primer-containing nucleic acid templates of the desired gene. Further steps include denaturing the primer-containing nucleic acid templates, contacting the promoter-containing primer with the denatured primer-containing nucleic acid templates to generate a plurality of promoter-containing double-stranded DNA templates (wherein the double-stranded nucleic acid templates are flanked with said promoter-containing primer in the 5′-end and said primer in the 3′-end of the other orientation) and transcribing the promoter-containing double-stranded DNA templates to form a plurality of amplified RNA sequences of said desired gene; so as to provide a clone of the desired gene in the form of RNA and promoter-containing double-stranded DNA templates.

Also provided is a method for determining the efficacy of a drug regiment against a gene or its cDNAs. This method includes generating an expression profile of cells which are treated by a drug, generating an expression profile of cells which are not treated by the drug, comparing the gene expression profile of the drug-treated cells to the gene expression profile of the untreated cells, by microarray and/or subtractive hybridizations and selecting the differently expressed genes from the compared gene expression profiles; so as to provide the gene and/or its cDNAs which are sensitive to the treatment of said drug.

Further novel features and other objects of the present invention will become apparent from the following detailed description, discussion and the appended claims, taken in conjunction with the drawings.

### BRIEF DESCRIPTION OF THE DRAWINGS

Referring particularly to the drawings for the purpose of illustration only and not limitation, there is illustrated:

FIG. 1 is an illustration of the preferred embodiment of RNA-polymerase chain reaction of the subject invention;

FIG. 2 is an illustration of second preferred embodiment of RNA-polynerase chain reaction of the subject invention;

FIG. 3 is an illustration of third preferred embodiment of the RNA-polymerase chain reaction of the subject invention; and

FIGS. 4a and 4b are the results of example 4 of the subject invention.

FIG. 5 is an illustration of the preferred embodiment of polymerase thermocycling reaction of the subject invention;

FIGS. 6a and 6b are the results of example 7 of the subject invention;

FIGS. 7a and 7b are the results of example 8 of the subject invention;

FIGS. 8a and 8b are the results of example 9 of the subject invention; and

FIG. 9 is the result of example 10 of the subject invention.

### DESCRIPTION OF THE PREFERRED EMBODIMENT

Although specific embodiments of the present invention will now be described with reference to the drawings, it should be understood that such embodiments are by way of example only and merely illustrative of but a small number of the many possible specific embodiments which can represent applications of the principles of the present invention. Various changes and modifications obvious to one skilled in the art to which the present invention pertains are deemed to be within the spirit, scope and contemplation of the present invention as further defined in the appended claims.

The present invention is directed to a novel polymerase cycling reaction method for mRNA amplification from single cells, named “RNA-polymerase chain reaction (RNA-PCR)”. Based on the proof-reading feature of an RNA polymerase activity (namely a transcriptional reaction), the RNA-PCR reaction provides much better lineage and higher fidelity of nucleic acid amplification than a traditional PCR-based reaction which uses DNA polymerase activities. A novel thermocycling procedure of the transcriptional amplification in conjunction with a reverse transcription activity is now provided by the current invention to increase the productivity of an RNA-PCR reaction. This method is primarily designed for differential screening of tissue-specific gene expressions at cell level, cloning full-length sequences of unknown gene transcripts, generating pure probes for hybridization assays, synthesizing peptides in vitro, and preparing complete mRNA/cDNA libraries for gene chip technology.

mRNA is the intermediate between a gene and the protein for which that gene codes. The more active a gene is the more mRNA will be produced. One method for determining the mechanism involved in a cell in a diseased state involves comparing the levels of mRNA expression between diseased tissues and normal tissues. An abnormal level of mRNA expression in a cell in a diseased state, as compared to a cell in a non-diseased state, is suggestive of an association of that mRNA with that disease. Once aberrant mRNA expression has been identified (i.e., via a gene chip), the thermocycling methods of the present invention will provide for the rapid isolation, purification, and amplification of the aberrant mRNA sequence, which can then be studied, using techniques well known to those skilled in the recombinant arts, to determine its mode of action and thereby develop a treatment to reduce and/or prevent its association with the diseased state of the cell.

Further, many diseased states are caused by mutations in a single gene, i.e., monogenic diseases like cystic fibrosis. These single gene mutations may lead to the production of abnormal mRNA sequences in a diseased cell, as compared to the mRNA sequence in a non-diseased cell. The methods of the present invention will allow for the rapid determination of abnormal mRNA sequence production of cells in the monogenic diseased state and allow for an efficient and expedited determination of a suitable treatment and or prevention (e.g., the delivery of a pharmaceutical composition comprising an antisense sequence to the abnormal mRNA). An additional advantage of the present invention is that it allows for the rapid production of high-fidelity mRNA/cDNA libraries that are useful in the analysis of oligo- and polygenic disorders as well as monogenic diseases. Hence, differential screening, in this regard will be fundamentally revolutionized by the methods of the present invention.

The methods of the present invention may further be of advantage to the development of the field of pharmocogenomics. Pharmacogenomics is a swiftly evolving field involved in the development of individualized medicine. As the human genome becomes more complete, the methods of the present invention can be used to increase the accuracy of and efficiency and expediency with which a database containing human sequence variations may be established. Such databases, along with the methods of the present invention, are fundamental to the development of personalized medicine.

The methods of the present invention allow for the rapid and accurate production of subject cell mRNA and cDNA libraries. Genes code for mRNAs. mRNAs code for proteins. Proteins participate in the causation of normal and diseased states. Hence, any one of these three elements are candidates for drug targeting. These libraries can be scanned to determine the efficacy of various drug regiments, either against mRNAs or against the proteins for which they code. Furthermore, individual differences in genes and their mRNAs, as well as the proteins they code for, may affect how well or poorly a person responds to a particular drug treatment. The methods of the present invention will greatly facilitate the prescreening of an individual's response to a drug with respect to their genetic makeup, hence allowing for the efficient prediction of possible side effects or efficiency a given drug might have on an individual due to that individual's genetic traits. For instance, enzymes are proteins that participate in the catabolic breakdown of various biological elements. Part of the efficacy of a drug has to do with how long it remains in a subject's system before being degraded by a given enzyme. The methods of the present invention will allow for the rapid production of mRNAs, and consequently the production of their resultant proteins, with which the efficacy of a given drug can be assayed, e.g., with regard to how rapidly it is broken down in the presence of the individual's related enzymes. This in turn will facilitate the determination of both what drugs and what pharmaceutical carriers should be used to effectuate the desired response.

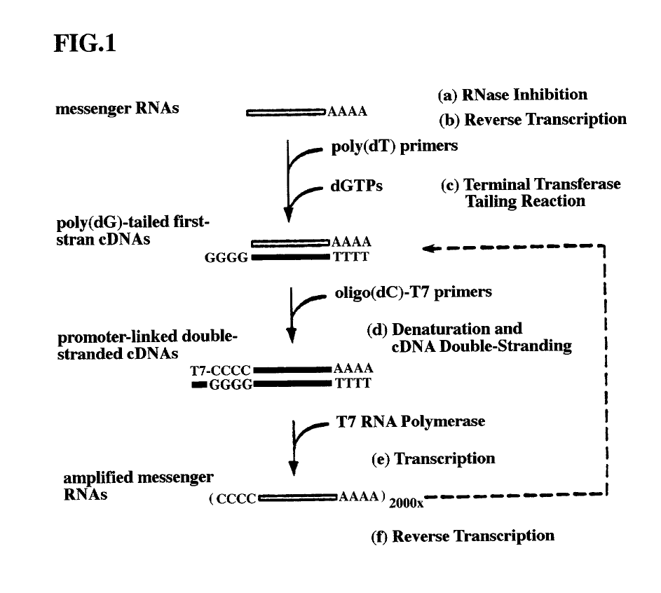

The method of the RNA-PCR includes repeating steps of reverse transcription, denaturation, double-stranded cDNA synthesis and in vitro transcription to bring up the population of mRNAs to two thousand fold in a single round of the above procedure. In brief, the preferred version (FIG. 1) of the present invention is based on: 1) prevention of mRNA degradation, 2) first reverse transcription and terminal transferase reaction to incorporate 3′-polynucleotide tails to the first-strand cDNAs, 3) denaturation and then double-stranded cDNA formation based on the extension of specific promoter-primers complementary to the 3′-polynucleotide tails, 4) transcription from a promoter to amplify mRNAs up to two thousand fold per round, and 5) repeating aforementioned steps to achieve desired RNA amplification.

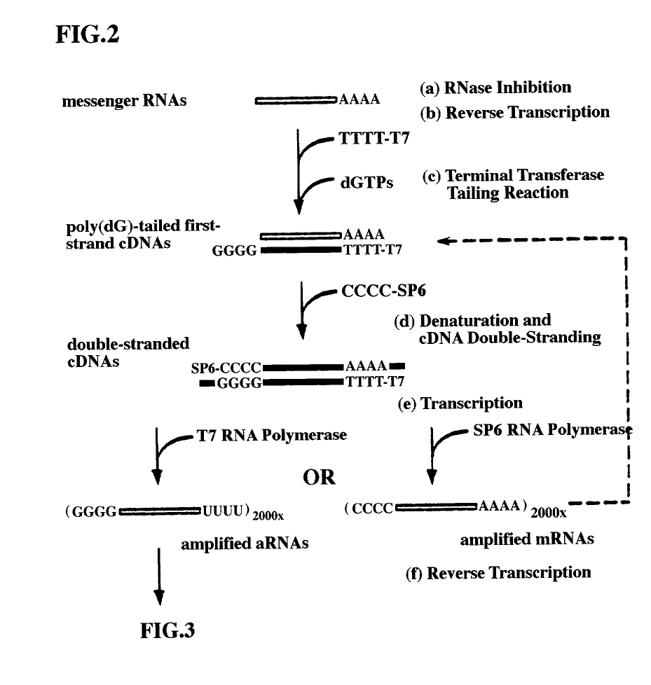

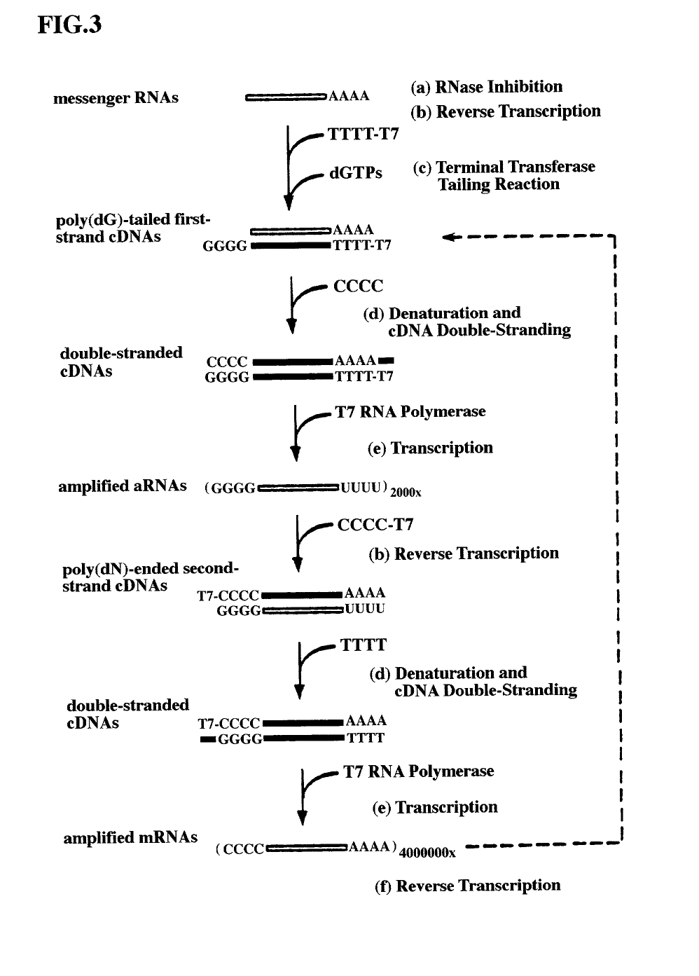

Alternatively, the second preferred version (FIG. 2) of the present invention is based on: 1) prevention of mRNA degradation, 2) first reverse transcription to incorporate first promoters to the 5′-ends of first-strand cDNAs and then addition of polynucleotide sequences to the 3′-ends of the first-strand cDNAs, 3) double-stranded cDNA synthesis based on the extension of second promoter sequences complementary to the 3 ′-polynucleotide regions of the first-strand cDNAs, 4) transcription to amplify either aRNAs or mRNAs up to two thousand fold in the first round of amplification cycle, and 5) repeating the aforementioned cycling steps to achieve the desired amount of RNAs. As shown in FIG. 2, the first promoter used here is different from the second promoter, resulting in the control of transcription by adding different RNA polymerases. The first promoter is incorporated for aRNA amplification, whereas the second promoter is designed for mRNA amplification. By this way in conjunction with a reverse transcription step, we can choose to amplify aRNAs, first-strand cDNAs, mRNAs or second-strand cDNAs of interest, depending on which RNA polymerase and nuclease we use. Although the second and third preferred embodiments (FIGS. 2 and 3) are more complicated than the first preferred embodiment (FIG. 1), the principle and broad features of the second and third preferred embodiments are completely within the scope of the first preferred embodiment of the present invention.

As used herein, the first-strand complementary DNA (cDNA) refers to a DNA sequence which is complementary to a natural messenger RNA sequence in an A-T and C-G composition. The antisense RNA (aRNA) refers to an RNA sequence which is complementary to a natural messenger RNA sequence in an A-U and C-G composition. And, the oligo(dT)-promoter sequence refers to an RNA polymerase promoter sequence coupled with a poly-deoxythymidylate (dT) sequence in its 3′-end, of which the minimal number of linked dT is seven; most preferably, the number is about twenty-six. The sense sequence refers to a nucleotide sequence which is in the same sequence order and composition as its homolog mRNA, whereas the antisense sequence refers to a nucleotide sequence which is complementary to its respective mRNA homologue. On the other hand, the oligo(antisense polynucleotide)-promoter sequence refers to an oligonucleotide sequence which is complementary to the polynucleotide-tail of said polynucleotide-tailed cDNAs and also linked to an RNA polymerase promoter in its 5′-end. And, Tth-like DNA polymerases refer to RNA- and DNA-dependent DNA polymerases with reverse transcription activity.

According to an embodiment of the present invention, a cycling procedure of reverse and in-vitro transcriptional reactions for amplifying intracellular mRNA populations is disclosed. This cycling procedure preferably starts from reverse transcription of intracellular mRNAs with Tth-like DNA polymerase, following a tailing reaction with terminal transferases and then denaturation of resulting mRNA-cDNA hybrid duplexes. After renaturation of the above tailed cDNAs to specific promoter-linked primers, double-stranded cDNAs are formed by Tth-like DNA polymerases. And then, promoter-specific RNA polymerase(s) is added to accomplish the transcriptional amplification of intracellular mRNAs. The novelties of this amplification cycling procedure of the present invention are as follows: 1) single copy rare mRNAs can be increased up to 2000 fold in one round of amplification without mis-reading mistakes, 2) the mRNA amplification is linear and does not result in preferential amplification of abundant mRNA species, 3) the mRNA degradation is inhibited by fixation and the addition of RNase inhibitors, and 4) the final mRNA products are of full-length and can be directly used to generate a complete cDNA library or synthesize proteins in vitro (Shi-Lung Lin et.al. Nucleic Acid Res. (1999) and Science (2001)).

In the second preferred embodiment, referring to FIG. 2, when the promoter of oligo(dT)-promoter primers is different from that of oligo(antisense polynucleotide)-promoter primers, the amplification of aRNAs and mRNAs can be separated by adding different RNA polymerases in the step (e), but not both. However, in the third preferred embodiment, referring to FIG. 3, when the promoter of oligo(dT)-promoter primers is the same as that of oligo(antisense polynucleotide)-promoter primers, the amplification of aRNAs and mRNAs must be separated by adding the same RNA polymerase in different steps, such as (e) and (h). Both preferred embodiments are capable of fulfilling the purpose of the present invention to amplify mRNAs from single cells. Although we currently need to add new RNA polymerase in every round of transcription due to the denaturation step, the finding of thermostable RNA polymerases may make the procedure of the present invention more convenient. For example, if thermostable RNA polymerases become available, the amplification cycle can be directly completed in a microtube by following the first preferred embodiment (FIG. 1) of the present invention. Examples as mentioned here will be developed into a continuation in part of the present invention and is not intended in any way to limit the broad features or principles of the present invention.

Instead of poly(dT), oligo(dT)-promoter, antisense polynucleotide and oligo(antisense polynucleotide)-promoter primers, we can use sequence-specific primers and sequence-specific promoter-linked primers to accomplish the amplification of normalized aRNAs, mRNAs, first-strand cDNAs and second-strand cDNAs of interest. The labeling of cDNAs is accomplished by incorporation of labeled nucleotides or analogs during reverse transcription of Tth-like DNA polymerase activity, while that of the RNAs is completed during transcription. The nucleotide sequences so generated are capable of being probes in a variety of applications, such as Northern blots, Southern blots, dot hybridization, in situ hybridization, position cloning, antisense knockout transfection and so on. Alternatively, the preferred embodiments (FIGS. 1, 2 and 3) provide amplified full-length mRNAs for in vitro translation. A cap-nucleotide can be added to the 5′-end of amplified mRNAs during the transcription step of the present invention. Unlike normalized RNAs, the capped mRNAs can be directly used in protein synthesis and may help the isolation of such a protein. The preferred cap-nucleotides include P¹-5′-(7-methyl)-guanosine-P³-5′-adenosine-triphosphate and P¹-5′-(7-methyl)-guanosine-P³-5′-guanosine-triphosphate.

On the other hand, the first step of the present invention can start from fixed cells as well as mRNAs; i.e., fixed cultured cells, frozen fresh tissues, fixed tissues or tissues in slides. Since this formed mRNAs are of full-length and carry RNA promoter regions for in vitro/vivo expression, the transfection of a certain gene transcript can be directly performed after its respective double-stranded cDNA is cloned into a competent vector. On the other hand, the present invention is also very useful in preparing complete full-length cDNA libraries for modem gene chip technology. Because the present invention is capable of generating a complete repertoire of full-length cDNAs from single cells, tissue-specific cDNA libraries based on special cell types can be formed and transferred onto a filter, membrane or chip for preserving these genetic information. As we all have different genetic information from a variety of major tissues and organs, the cDNA-encoded gene chips may function as an individual source for differential screening, pathological diagnosis, physiological prognosis and genetic identification. Thus, the methods of the present invention will advance the establishment of cDNA libraries of tissues at different stages of development, facilitating our understanding of both normal and aberrant development. Further, these methods will advance the expansion of our understanding of genomic libraries derived from other species, especially as they relate to a determination of mRNA derived protein function.

Further still, the methods of the present invention will advance, not only the development of medicine directed to the individual but also facilitate the development of an understanding of genetic variation, on the individual as well as on the genetic, allelic, level allowing for the generation of drugs specifically targeted to allelic variants. Finding out not only allelic variants but variations among individuals will also revolutionize FDA clinical trials as they will then be focused on administering drugs only to that sample set whose genetic makeup has been predetermined to be benefited from the use of the newly developed drug. These kinds of approaches will become more and more important following the completion of the human genome project.

In the preferred embodiments (as shown in FIGS. 1, 2 and 3) of the present invention, according to the high amplification rate of RNA polymerase (about 2000 fold/cycle), the labor- and time-consuming factors in this RNA-PCR can be reduced to the minimum. Also, the preparation of amplified mRNAs is cheaper and more efficient than poly(dT)-linked chromatography columns in previous methods. Most importantly, this RNA amplification can be carried out in microtubes with only a few cells. Taken together, these special features make the current content of RNA-PCR as simple, fast, and inexpensive as a kit for concisely isolating amplified mRNA sequences of interest.

This method may use a primer and promoter-containing primer that is of either gene-specific sequences or random sequences (i.e., a random hexamer). This method may be used for the purposes of producing an amplified genomic DNA sequence or sequences for a specific gene or genes for the whole genome genomic DNAs. This is useful for producing amplified DNA sequence or sequences that can be utilized in a genotyping scheme, i.e., the generation of a genomic DNA profile. (See Example 8 for more details.) Such a scheme is useful for screening and or analyzing different gene sequences within an individual's genome. By practicing the methods of the present invention a comparison of different genomic DNA profiles may be made.

In a further preferred embodiment, is provided a method for the differential screening of tissue specific gene expression at a cellular level. This method includes the steps of:

a. generating a gene expression profile, as described above, of cells which are dissected from a homogeneous cell region of a tissue sample by a single-cell isolation device selected from the group including shredder columns, micromanipulators and laser-capture devices;

b. comparing the gene expression profile of the cells to another gene expression profile by microarray and/or subtractive hybridizations; and

c. selecting the differently expressed genes from the compared gene expression profiles; so as to provide a differentially expressed gene profile of the tissue cells.

Furthermore, a method for preparing labeled RNA/DNA probes for a gene chip technology, is also provided. This method includes the steps of:

a. generating a mRNA library, as described above, of cells of interest;

b. combining said mRNA library with a primer, a promoter-containing primer, a kind of labeled deoxynucleotide or ribonucleotide triphosphates (i.e., labeled by a chemical selected from the following biotin, avidin, digoxigenin fluorescein, Cy3, Cy5 and radioactive isotopes), a plurality of unlabeled deoxynucleotide and ribonucleotide triphosphates, a reverse transcription enzyme, a DNA-dependent DNA polymerase enzyme and an RNA polymerase enzyme;

c. contacting said promoter-containing primer with the RNA templates of said mRNA library to generate a plurality of promoter-containing nucleic acid templates;

d. denaturing the promoter-containing nucleic acid templates;

e. contacting the primer with the denatured promoter-containing nucleic acid templates to generate a plurality of promoter-containing double-stranded DNA templates, wherein the double-stranded nucleic acid templates are flanked with said promoter-containing primer in one end and said primer in the other end of the other orientation;

f. transcribing the promoter-containing double-stranded DNA templates to form a plurality of labeled RNA probes;

g. reverse-transcribing said RNA probes to form a plurality of labeled DNA probes; and

h. contacting said labeled RNA or DNA probes with a gene chip or microarray; so as to provide an expression profile of the interested cells.

The present invention also includes a method for cloning full-length sequences of unknown gene transcripts, which includes the steps of:

a. denaturing a plurality of nucleic acid templates containing a gene or its transcript which is desired to be cloned;

b. combining said denatured nucleic acid templates with a primer complementary to the 3′-end of said desired gene, a promoter-containing primer homologous to the 5′-end of said desired gene, a plurality of deoxynucleotide triphosphates, a plurality of ribonucleotide triphosphates, a reverse transcription enzyme, a DNA-dependent DNA polymerase enzyme and an RNA polymerase enzyme;

c. contacting said primer with the nucleic acid templates to generate a plurality of primer-containing nucleic acid templates of said desired gene;

d. denaturing the primer-containing nucleic acid templates;

e. contacting the promoter-containing primer with the denatured primer-containing nucleic acid templates to generate a plurality of promoter-containing double-stranded DNA templates, wherein the double-stranded nucleic acid templates are flanked with said promoter-containing primer in the 5′-end and said primer in the 3′-end of the other orientation; and

f. transcribing the promoter-containing double-stranded DNA templates to form a plurality of amplified RNA sequences of said desired gene; and so as to provide a clone of the desired gene in the form of RNA and promoter-containing double-stranded DNA templates.

This method as well may use a primer and promoter-containing primer that is of either gene-specific sequences or random sequences (i.e., a random hexamer) and this method may also be used for the purposes of producing an amplified genomic DNA sequence or sequences for a specific gene or genes for the whole genome genomic DNAs. This, again, is useful for producing amplified DNA sequence or sequences that can be utilized in a genotyping scheme.

Also provided is a method for determining the efficacy of a drug regiment against a gene or its cDNAs, which includes the steps of:

a. generating an expression profile, as described above, of cells which are treated by a drug;

b. generating an expression profile, as described above, of cells which are not treated by said drug;

c. comparing the gene expression profile of the drug-treated cells in step (a) to the gene expression profile of the untreated cells in the step (b) by microarray and/or subtractive hybridizations; and

d. selecting the differently expressed genes from the compared gene expression profiles; so as to provide the gene and/or its cDNAs which are sensitive to the treatment of said drug.

Although certain preferred embodiments of the present invention have been described, the spirit and scope of the invention is by no means restricted to what is described above. For example, within the general framework of: a) one or more specific primers for reverse transcription and polymerase extension reaction; b) one or more RNA promoter-primers for transcription; c) seven or more deoxynucleotides extended in a promoter-linked polynucleotide primer; d) seven or more of the same nucleotides added to the 3′-end of the first-strand cDNAs; e) one or more rounds of the cycling steps for RNA amplification, there is a very large number of permutations and combinations possible, all of which are within the scope of the present invention.

### EXAMPLE 1

### Cell Fixation and Permeabilisation

LNCaP cells, a prostate cancer cell line, were grown in RPMI 1640 medium supplemented with 2% fetal calf serum. One 70% confluent 60 mm dish culture was trypsinized, collected and washed three times in 5 ml phosphate buffered saline (PBS, pH 7.2) at room temperature, then suspended in 1 ml of ice-cold 10% formaldehyde solution in 0.15M NaCl. After one hour incubation on ice with occasional agitation, the cells were centrifuged at 13,000 rpm for 2 minutes and washed three times in ice-cold PBS with vigorous pipetting. The collected cells were resuspended in 0.5% non-ionic detergent (octylphenoxy)polyethanol and incubated for one hour with frequent agitation. After that, three washes were given to cells in ice-cold PBS containing 0.1M glycine and the cells were resuspended in 1 ml of the same buffer with vigorous pipetting in order to be evenly separated into small aliquots and stored at −70° C. for up to a month.

### EXAMPLE 2

### First Reverse Transcription and Polynucleotide Tailing of The First-Strand cDNAs

For the first reverse transcription of mRNAs in cells, one hundred of the fixed cells were thawed, resuspended in 20 micoliter (μl) of DEPC-treated ddH₂O, mixed with 25 pmol oligo(dT)-T7 promoter (SEQ ID.1), heated to 65° C. for 5 minutes and then cooled on ice. A 50 μl RT reaction was prepared, comprising 10 μl of 5×Mg-containing RT buffer (Boehringer Mannheim), dNTPs (1 mM each for dATP, dGTP, dCTP and dTTP), RNase inhibitor and above the cooled cells. After C. therm. polymerase (5U) was added, the RT reaction was mixed and incubated at 55° C. for 10 minutes and shifted to 72° C. for one hour, and then the cells were washed once with PBS and resuspended in a 50 μl tailing reaction, comprising 2 mM dGTP, 10 μl of 5× tailing buffer (250 mM KCl, 50 mM Tris-HCl, 8 mM MgCl₂, pH 8.3 at 20° C.). The tailing reaction was heated at 94° C. for 3 minutes and then chilled in ice for mixing with terminal transferase (20U), following further incubation at 37° C. for 20 min. This formed said polynucleotide-tailed first-strand cDNAs.

### EXAMPLE 3

### Denaturation, Double-Stranded cDNA Synthesis and Transcriptional Amplification

The above tailing reaction was stopped at 94° C. for 2 minutes, mixed with 25 pmol poly(dC) primer (SEQ ID.2), and denatured at 94° C. for 3 more minutes. After 1 minute centrifuging at room temperature, 1 mM dNTPs and C. therm. polymerase (5U) were added to form double-stranded cDNAs at 70° C. for 5 minutes. To increase the amount of desired RNAs, the T7 promoter-linked first-strand cDNA served as a coding strand for the transcription of T7 RNA polymerase. As few as several cells in 5 μl of the above resulting reaction can be used to accomplish full-length aRNA amplification during the following reaction. A transcription reaction (50 μl) was prepared, containing 5 μl of 10×transcription buffer (Boehringer Mannheim), rNTPs (2 mM each for ATP, GTP, CTP and UTP), RNA inhibitor and T7 RNA polymerase (2000U). After three hour incubation at 37° C., the cDNA transcripts were isolated from both cells and supernatant, and can be directly used in the following reverse transcription. The reaction was finally stopped at 94° C. for 3 minutes and chilled in ice immediately.

### EXAMPLE 4

### Second Reverse Transcription, Denaturation

### Double-Stranded cDNA Synthesis and mRNA Amplification

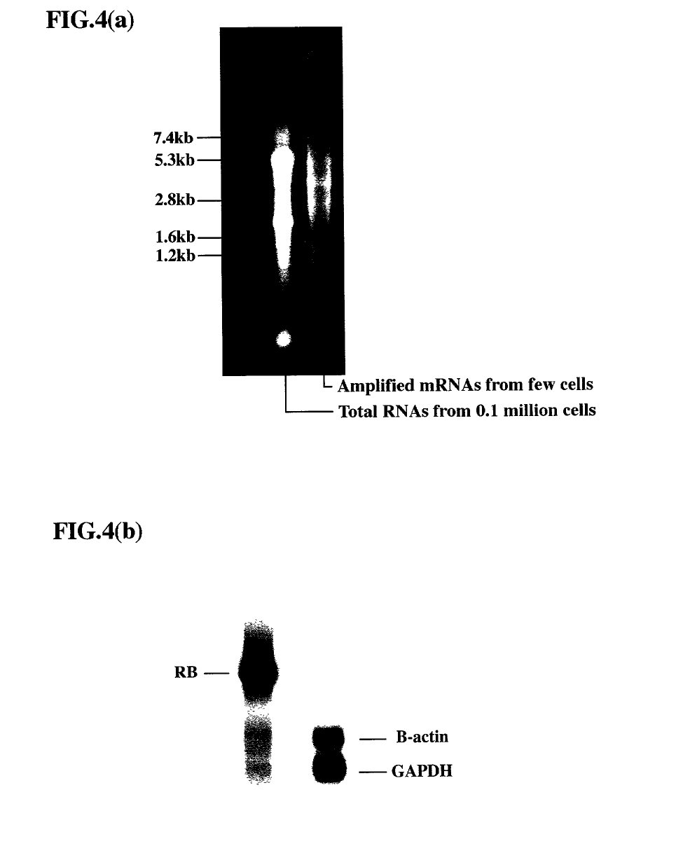

A 50 μl RT reaction was prepared, comprising 10 μl of 5×Mg-containing RT buffer (Boehringer Mannheim), 25 pmol oligo(dC)-SP6 promoter primer (SEQ ID.3), 2 mM dNTPs, RNase inhibitor and 5 μl of above aRNA-containing supernatant. After C. therm. polymerase (5U) was added, the RT reaction was mixed and incubated at 55° C. for 10 minutes and shifted to 72° C. for one hour. This formed said second-strand cDNAs. After another denaturation at 94° C. for 3 minutes and mixing with 25 pmol poly(dT) primer (SEQ ID.4) at room temperature for 1 min, double-stranded cDNAs can be formed by adding 1 mM dNTPs at 70° C. for 5 min. A transcription reaction (50 μl) was then prepared to generate said amplified mRNAs, containing 5 μl of 10×transcription buffer (Boehringer Mannheim), 2 mM rNTPs, RNA inhibitor and SP6 RNA polymerase (2000U). After three hour incubation at 37° C., the cDNA transcripts were isolated and can be directly used in another round of RNA-PCR. The final reaction was stopped at 94° C. for 3 minutes and chilled in ice immediately. The quality of the final amplified mRNAs (2 μg) was assessed on a 1% formaldehyde-agarose gel, ranging from 500 bp to above 10 kb (FIG. 4a). We also have successfully identified the gene transcripts of RB, β-actin and GAPDH on the Northern blots of full-ranged LNCaP mRNAs made by the present invention (FIG. 4b).

### EXAMPLE 5

### Cycling Procedure of RNA Amplification

Few fixed cells were applied to a reverse transcription reaction (50 μl) on ice, comprising 10 μl of 5×RT&T buffer (100 mM Tris-HCl, pH8.3 at 25° C., 600 mM KCl, 300 mM (NH₄)₂SO₄, 40 mM MgCl₂, 5M betaine, 35 mM DTE and 10 mM spermidine), 1 μM poly(dT) primer (SEQ ID.4), dNTPs and RNase inhibitors (10U). After C. therm. polymerase (6U) was added, the reaction was incubated at 52° C. for 3 minutes and shifted to 65° C. for another 30 minutes. The first-strand cDNAs so obtained were collected in a tailing reaction (50 μl), comprising 10 μl of 5×tailing buffer (250 mM KCl, 100 mM Tris-HCl, 4 mM CoCl₂, 10 mM MgCl₂, pH8.3 at 20° C.) and 0.5 mM dGTP. After terminal transferase (75U) was added, the reaction was incubated at 37° C. for 15 minutes, stopped by denaturation at 94° C. for 2 minutes and mixed with 1 μM oligo(dC)₁₀-T7 promoter primer (SEQ ID.5). Taq DNA polymerase (3.5U) and dNTPs were then added to double-strand the above tailed cDNAs at 52° C. for 3 minutes and then 72° C. for 7 minutes. A transcription reaction (50 μl) was prepared, containing 10 μl of 5×RT&T buffer, 2 mM rNTPs, RNA inhibitors (10U), T7 RNA polymerase (200U) and the promoter-linked double-stranded cDNAs. After one hour incubation at 37° C., the mRNA transcripts were isolated and used directly for the next round of RNA-PCR without the tailing reaction, containing 10 μl of 5×RT&T buffer, 1 μM poly(dT) primers, 1 μM oligo(dC)₁₀-promoter primers, 2 mM dNTPs, 2 mM rNTPs, T7 RNA polymerase, C. therm. polymerase, Taq DNA polymerase and the mRNA products. After two cycles of transcriptional amplification, the quality of mRNA products can be assessed on a 1% formaldehyde-agarose gel (Shi-Lung Lin et.al. Nucleic Acid Res. (1999)).

Example 1 describes a preferred step for the prevention of intracellular RNA/DNA degradation before the step (a) of the present invention. The Examples 2-4 are directed to different applications of the present invention. Since previous methods failed to provide linear amplification due to a failure of the PCR reaction through high (dG/dC)-content sequences, our invention overcomes this bottleneck by designing special promoter-containing primers (SEQ ID.2, 4 and 5) which anneal to the 5′-end of a desired nucleic acid template for transcriptional amplification. Linear amplification is a natural property of RNA polymerase activity (Van Gelder et.al., Proc. Natl. Acad. Sci. USA 87: 1663-1667 (1990)). Although commercialized buffers for each enzyme activity are slightly different, we have also designed a series of novel RT&T buffers (as shown in Examples 2-4) to unify the optimized conditions for the thermocycling procedure of the present invention. The temperatures are changed to fulfill the maximal activities of each enzymatic step, depending on the individual property of enzymes. The present invention is useful when linear amplification is required or only picogram starting materials can be acquired for RNA/DNA amplification. Its results not only provide a molecular diagnosis of diseased genes in vivo but also increase the resolution of current genetic research to a single-cell scale. Alternatively, the present invention is directed to a novel polymerase thermocycling reaction method for nucleic acid amplification, named “RNA-polymerase cycling reaction (RNAPCR)”. This method is primarily designed for differential screening of tissue-specific gene expressions at cell level, cloning full-length sequences of unknown gene transcripts, generating pure probes for hybridization assays, synthesizing peptides in vitro, and preparing complete mRNA/cDNA/genome libraries for current genechip/microarray technologies. Hence, the methods of the present invention may also be useful in protein pathway profiling. Being able to rapidly produce a linear representation of a cell's mRNA will allow for a more representative protein model for the use in determining protein-protein interactions within and between cells. The mRNAs, thus produced, may be transfected into single, or multiple, cell organisms for the determination of how the proteins in question function in that primitive cell organism, both individually and in relation one to the other. Using the present invention with model organisms is a fast and cost efficient way of studying biological systems and identifying new protein targets for drug development.

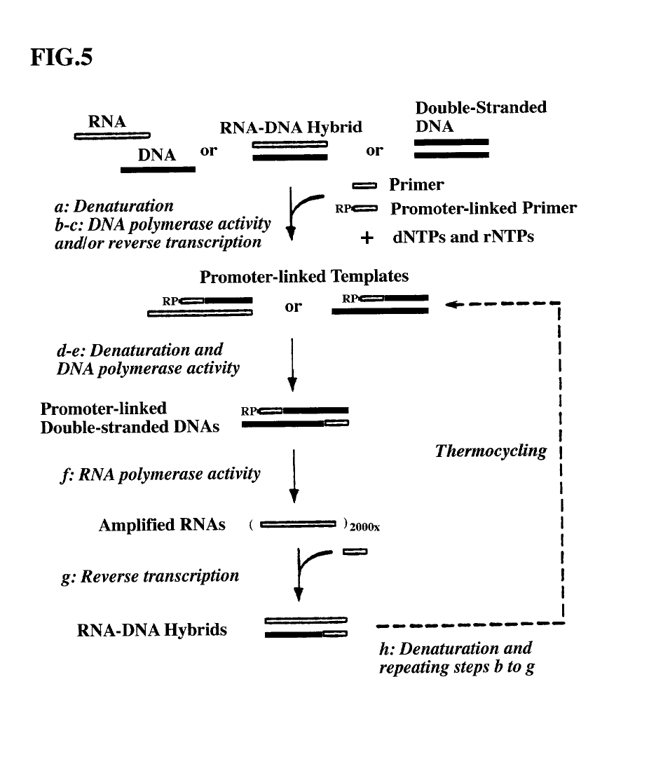

The principle of RNAPCR relies on the repeating steps of reverse transcription, denaturation, double-stranded DNA synthesis and in-vitro transcription at different optimized temperatures to bring up the population of desired sequences to two thousand fold in one cycle of the above procedure. In brief, the preferred version (FIG. 5) of the present invention is based on: 1) denaturation of the desired nucleic acid templates, 2) reverse transcription and/or DNA polymerization to incorporate an RNA promoter into the nucleic acid templates, 3) denaturation and then double-stranding the DNA templates, 4) in-vitro transcription from the promoter region to generate RNA sequences, and 5) repeating the aforementioned steps (2)-(4) to achieve the desired RNA amplification.

We improve an RNA polymerase thermocycling procedure which is previously designed for amplifying a full spectrum of intracellular full-length mRNAs from single cells (Lin et.al. Nucleic Acid Res. 27: 4585-4589 (1999)). As shown, similar to Example 7, this improved thermocycling procedure preferably starts from reverse transcription of intracellular mRNAs with Tth-like thermostable polymerases, such as reverse transcription activity of C. therm. polymerase which is initiated with primers (SEQ ID.6 or 8) at about 65˜72° C. for about 30 minutes. Additionally, a (dG/dC)-tailing reaction with terminal transferases can be performed at about 37° C. for about 15 minutes before denaturation of the resulting RNA-DNA hybrids. After re-annealing of the above DNAs to a promoter-linked primer (SEQ ID.7 or 9), double-stranded DNA templates are formed by the same Tth-like thermostable polymerases. And then, a promoter-specific RNA polymerase(s) is added to accomplish the transcriptional amplification of intracellular mRNAs. The novelties and advantages of this first preferred embodiment are as follows: 1) rare RNA species can be increased up to 2000 fold in one round of amplification without mis-reading mistakes, 2) the transcriptional amplification is linear and does not result in preferential amplification of abundant RNA species, 3) the RNA degradation by RNase is decreased by thermal cycling conditions, and 4) the full-length constructs of RNAs can be maintained for generating a complete cDNA library or synthesize proteins/peptides in vitro.

Alternatively, the present invention is an improved thermocycling procedure for preparing amplifiable nucleic acid fragments which represent a whole genome library. As shown in Example 8, this second preferred embodiment preferably starts from the synthesis of promoter-incorporated double-stranded DNA templates with Tth-like thermostable polymerases, such as reverse transcription and DNA polymerization activities of C. therm. polymerase which is initiated with sequence-specific primers and promoter-containing primers (SEQ ID.8 and 9) at about 68° C. for about 30 minutes. Denaturation of the starting genomic DNAs is performed at about 94° C. for about 5 minutes before the synthesis of promoter-incorporated double-stranded DNA templates. And then, a promoter-specific RNA polymerase(s) is added to accomplish the transcriptional amplification of genomic fragments. The novelties and advantages are similar to the aforementioned as follows: 1) high (dG/dC)-content genome fragments can be faithfully amplified with high fidelity, 2) the transcriptional amplification is linear and does not result in the preferential amplification as previous PCR-based methods, 3) the nucleic acid degradation by RNase/DNase is decreased by thermal cycling conditions, and 4) a full spectrum of genome libraries can be obtained by differently designed primers which are annealing to specific genomic sequences of interest.

Instead of SEQ ID.6-9, we can use sequence-specific primers and sequence-specific promoter-linked primers to detect a specific RNA, DNA, genomic sequence of interest. As shown in Example 9, the labeling of resulting DNAs is achieved by the incorporation of labeled nucleotides or analogs during reverse transcription, while that of the resulting RNAs is accomplished during in-vitro transcription. Such an approach provides molecular detection with high resolution and fidelity for genetic research and in-vitro diagnosis. Also, these labeled nucleic acid sequences can serve as probes in a variety of biochemical applications, such as Northern blot, Southern blot, dot blot and genechip/microarray hybridization, and in situ hybridization, position cloning detection and antisense knockout transfection. Alternatively, this third preferred embodiment (Example 9) provides amplified mRNAs with a start codon for in-vitro translation. A cap-nucleotide is added to the 5′-end of amplified mRNAs during the transcriptional amplification step of the present invention. These capped mRNAs can be directly used in protein/peptide synthesis, and may help the generation of antibodies against such proteins as well as proteomic analysis (proteinchip). A preferred cap-nucleotide includes P¹-5-(7-methyl)-guanosine-P³-5′-adenosine-triphosphate and/or P¹-5′-(7-methyl)-guanosine-P³-5′-guanosine-triphosphate.

More flexibly, the present invention can start from fixed cells (Examples 7 and 8) as well as nucleic acid sequences (Example 9); i.e., fixed cultured cells, frozen fresh tissues, fixed tissues or tissues in slides. Since the cDNA sequences so obtained are potentially of full-length and carry an RNA promoter in their 5′-end, the overexpression transfection of a certain gene can be performed directly to cells either in vitro or in vivo. On the other hand, the present invention is also very useful for preparing single-stranded RNA/DNA probes for genechip/microarray technologies and/or preparing peptide products for proteinchip analysis. Because the present invention is capable of generating a complete repertoire of full-length mRNAs from single cells, a tissue-specific gene/peptide library can be further obtained and re-amplified for preserving these genetic information without tedious sample collections from tested animals and patients. As we all have slightly different genetic information, the present invention may provide a non-invasive collection method and unlimited nucleic acid samples for many molecular biological analysis, such as differential screening, pathological diagnosis, physiological prognosis and genetic identification. This kind of approach will become more and more important following the completion of the human genome project.

In the preferred embodiments (Examples 7-9) of the present invention, according to the high linear amplification rate of RNA polymerases (up to 200 fold/unit), the labor- and time-consuming factors in this thermocycling method can be reduced to the minimum. Also, the transcriptional amplification of RNA/DNA/genomic sequences is much easier and cheaper than traditional extraction methods. Most importantly, the present invention can be carried out in a microtube with picogram amounts of samples, providing unlimited and amplifiable nucleic acid resources for current genetic research and non-invasive diagnosis with a much higher standard of humanity. Taken together, these special features make the content of the present invention as simple, fast, and reliable as a kit for concisely amplifying and detecting the desired sequence(s) of interest.

Although certain preferred embodiments of the present invention have been described, the spirit and scope of the invention is by no means restricted to what is described above. For example, within the general framework of: a) one or more kinds of nucleic acid templates; b) one or more kinds of primers for reverse transcription and DNA polymerization reaction; c) one or more promoter-containing primers for in-vitro transcription; d) one or more buffered conditions for maintaining said enzyme activities; e) less or more steps of the thermocycling procedure of the present invention, there is a very large number of permutations and combinations possible, all of which are within the scope of the present invention.

### EXAMPLE 6

### Cell Fixation and Permeabilisation

LNCaP cells, a prostate cancer cell line, were grown in RPMI 1640 medium supplemented with 10% fetal calf serum. One 70% confluent 60 mm dish culture was trypsinized, collected and washed three times in 5 ml phosphate buffered saline (PBS, pH 7.2) at room temperature, then suspended in 1 ml of ice-cold 10% formaldehyde solution in 0.15M NaCl. After one hour incubation on ice with occasional agitation, the cells were centrifuged at 13,000 rpm for 2 minutes and washed three times in ice-cold PBS with vigorous pipetting. The collected cells were resuspended in 0.5% non-ionic detergents, such as (octylphenoxy)-polyethanol or polyoyethylenesorbitan, and incubated for one hour with frequent agitation. After that, three washes were given to cells in ice-cold PBS containing 0.1M glycine and the cells were resuspended in 1 ml of the same buffer with vigorous pipetting for even separation into small aliquots for storage at −70° C. for up to a month.

### EXAMPLE 7

### Thermocycling Amplification of A cDNA Library from Single Cells

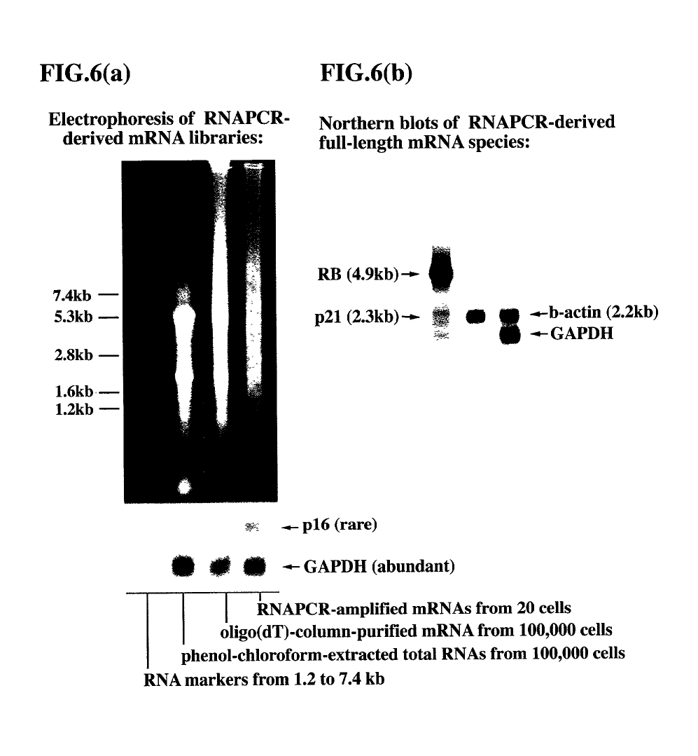

A few fixed LNCaP cells were applied to a starting solution (20 μl ) on ice, comprising 2 μl of 10×RT&T buffer (400 mM Tris-HCl, pH 8.3 at 25° C., 300 mM KCl, 60 mM MgCl₂, 2M betaine, 100 mM DTE and 20 mM spermidine), 1 μM poly(dT)₂₄ primer (SEQ ID.6), deoxynucleotide triphosphates (2 mM each for dATP, dCTP, dGTP and dTTP), ribonucleotide triphosphates (2 mM each for ATP, CTP, GTP and UTP) and RNase inhibitors (10U). After AMV reverse transcriptase (6U) was added, the reaction was incubated at 42° C. for 50 minutes and shifted to 52° C. for another 10 minutes. The resulting cDNAs so obtained were diluted to 50μl by molecular grade water, and terminal transferase (75U) and dGTP (2 mM) were added to form high (dG)-content tails to the cDNAs at 37° C. for 15 minutes. After denaturation at 94° C. for 3 minutes, 1 μM oligo(dC)₁₀N-T7 promoter primers (SEQ ID.7) and Taq DNA polymerase (3.5U) were added to double-strand the tailed cDNAs at 52° C. for 3 minutes and then 72° C. for 7 minutes. A transcriptional amplification was then performed by adding 3 μl of 10×RT&T buffer and T7 RNA polymerase (160U). After incubation at 37° C. for 60 minutes, the RNA transcripts were generated and can be used for the next cycle of the above procedure without the tailing reaction. The quality of amplified RNA products can be observed on a 1% formaldehyde-agarose gel as shown in FIG. 6a. Northern blots of p21 and RB gene transcripts can be detected in their full-length sizes as shown in FIG. 6b.

### EXAMPLE 8

### Thermocycling Amplification of A Genomic DNA Library from Single Cells

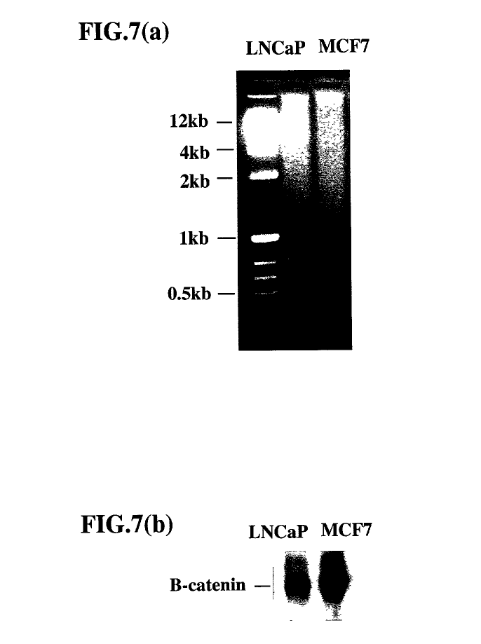

Fresh MCF7 cells were boiled at 94° C. for 5 minutes to break down both cellular and nuclear membranes as well as to denature genomic DNAs, and then applied to a starting solution (50 μl) on ice, comprising 5 μl of 10×RT&T buffer (400 mM Tris-HCl, pH 8.3 at 25° C., 500 mM NaCl, 300 mM (NH4)2SO4, 80 mM MgCl₂, 2M betaine, 100 mM DTE and 20 mM spermidine), 1 μM random 10mer primers (SEQ ID.8), 1 μM SP6 promoter-containing degenerate primers (SEQ ID.9), deoxynucleotide triphosphates (2 mM each), ribonucleotide triphosphates (2 mM each), and C. therm./Taq polymerase mixture (6U/3.5U). The reaction was performed following a thermocycling procedure at 52° C. for 3 minutes, 65° C. for 30 minutes, 94° C. for 3 minutes, 52° C. for 3 minutes, 68° C. for 10 minutes, and then 37° C. for 60 minutes after adding SP6 RNA polymerase (100U), and another cycle of amplification can be iterated as aforementioned. This formed a spectrum of amplified RNA/DNA sequences which represent a whole genome library. The quality of such amplified RNA/DNA products can be observed on a 1% TBE agarose gel as shown in FIG. 7a. Southern blot of β-catenin gene can be detected to be tetraploid as shown in FIG. 7b.

### EXAMPLE 9

### Detection of A Specific Nucleic Acid Sequence in An RNA/cDNA Library

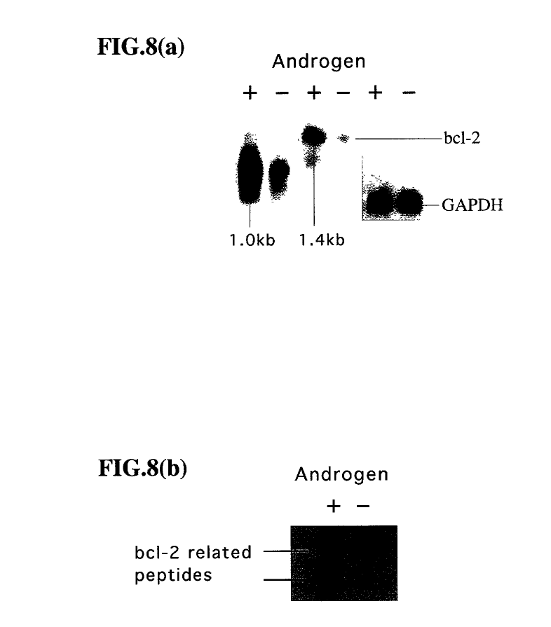

The resulting RNA/cDNA library (10 pg) from Example 2 was denatured at 94° C. for 2 minutes and applied to a starting solution (50 μl) on ice, comprising 5 μl of 10×RT&T buffer (400 mM Tris-HCl, pH 8.3 at 25° C., 400 mM KCl, 80 mM MgCl₂, 2M betaine,100 mM DTE and 20 mM spermidine), 1 μM antisense-bcl2 primers (SEQ ID.10), 1 μM T7 promoter-containing sense-bcl2 primers (SEQ ID.11), deoxynucleotide triphosphates (2 mM each for dCTP dGTP, dTTP and 3000 Ci/mM P³²-labeled dATP), ribonucleotide triphosphates (2 mM each), cap-nucleotides, and M-MuLV reverse transcriptase/Pwo polymerase mixture (6U/3.5U). The reaction was performed following a thermocycling procedure at 37° C. for 50 minutes, 94° C. for 3 minutes, 52° C. for 3 minutes, and then 37° C. for 60 minutes after adding Klenow/T4 DNA polymerase mixture (3U each), T7 RNA polymerase (160U) and M-MuLV reverse transcriptase (6U), and another cycle of amplification can be reiterated for generating enough amount of desired bcl-2 RNA sequences. The amplified bcl-2 RNAs (10 μl) were added to an in-vitro translation reaction (rabbit cell lysate extractions with RNase inhibitors) and incubated at 30° C. for 30 minutes. As shown in FIGS. 8a and 8b, the amplified bc1-2 RNA and peptide products were only detected by androgen-retrieval from LNCaP cells on a 6% polyacrylamide gel, indicating the immortal and tumorigenic property of these metastatic cancer cells (Shi-Lung Lin et.al. Science (2001)).

Example 6 describes a preferred step for the prevention of intracellular RNA/DNA degradation before the step (a) of the present invention. Examples 7-9 are directed to different applications of the present invention. Since previous methods failed to provide linear amplification due to the failure of PCR reaction through high (dG/dC)-content sequences, our invention overcomes this bottleneck by designing special promoter-containing primers (SEQ ID.7, 9 and 10) which anneal to the 5′-end of a desired nucleic acid template for transcriptional amplification. Linear amplification is a natural property of RNA polymerase activity (Van Gelder et.al., Proc. Natl. Acad. Sci. USA 87: 1663-1667 (1990)). Although commercialized buffers for each enzyme activity are slightly different, we have also designed a series of novel RT&T buffers (as shown in Examples 7-9) to unify the optimized conditions for the thermocycling procedure of the present invention. The temperatures are changed to fulfill the maximal activities of each enzymatic step, depending on the individual property of enzymes. The present invention is useful when linear amplification is required or only picogram starting materials can be acquired for RNA/DNA amplification. Its results not only provide a molecular diagnosis of diseased genes in vivo but also increase the resolution of current genetic research to the single-cell scale.

### EXAMPLE 10

### Microarray Hybridization

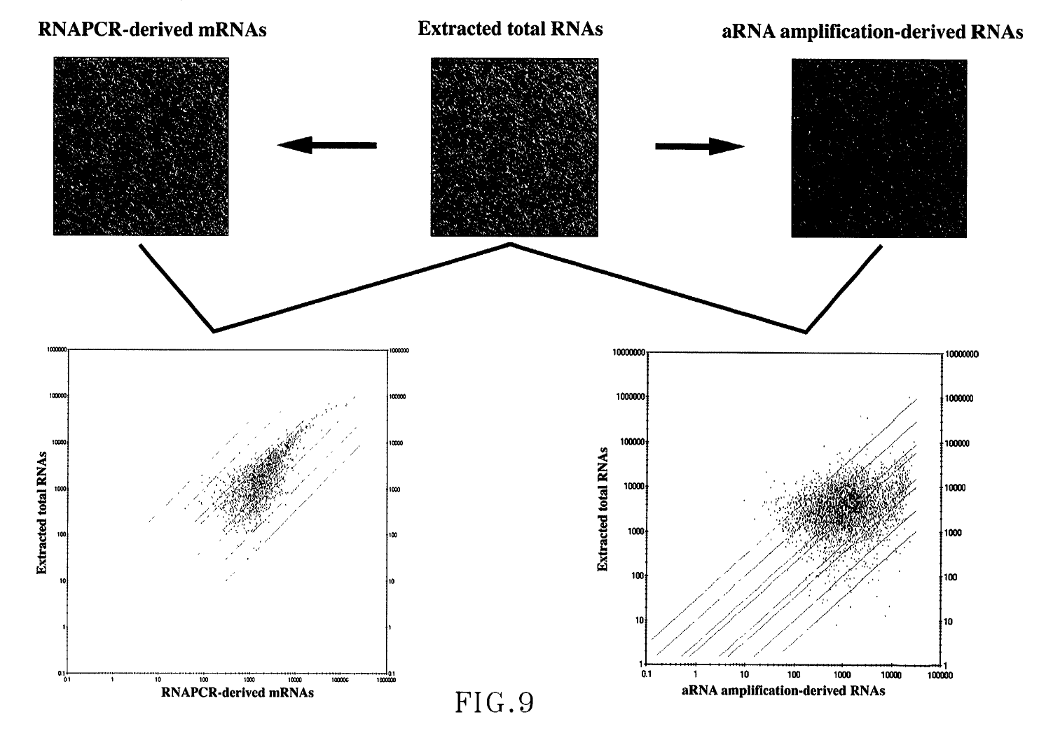

A set of four oligonucleotide microarrays (GeneChip U95 A2 arrays, Affymetrix, Santa Clara, Calif.) containing total 12,600 genes are used for hybridization. Hybridizations are completed in 200 μl of AFFY buffer (Affymetrix) at 40° C. for 16 hr with constant mixing. After hybridization, arrays are rinsed three times with 200 μl of 6×SSPE-T buffer (1×0.25 M sodium chloride/15 mM sodium phosphate, pH 7.6/1 mM EDTA/0.005% Triton) and then washed with 200 μl of 6×SSPE-T for 1 hr at 50° C. The arrays are rinsed twice with 0.5×SSPE-T and washed with 0.5×SSPE-T at 50° C. for 15 min. Staining is done with 2 μg/ml streptavidin-phycoerythrin (Molecular Probes) and 1 mg/ml acetylated BSA (Sigma) in 6×SSPE-T (pH 7.6). The arrays are read at 7.5 μm with a confocal scanner (Molecular Dynamics) and analyzed with GeneChip software, version 4.0. The samples are normalized by using the total average difference between perfectly matched probe and the mismatched probe. The differential signals that are induced greater than 4-fold are collected. The 4-fold is arbitrarily chosen as the cut-off and is more conservative than the cut-off (2-fold) recommended by Affymetrix. The comparison of microarray results between traditional aRNA and RNAPCR-derived RNA probes from the same sample is shown by FIG. 9. A highly linear correlation of gene coverage is found in the abundant (red) and moderate (blue) mRNA species of both probe preparations, indicating a very good reproducibility of both methods. However, the RNAPCR-derived RNA probes are amplified from about twenty cells as shown in Example 11, while the traditional aRNA probes are prepared from 2 μg total RNAs as shown in Example 12.

### EXAMPLE 11

### RNA Probe Preparation by RNA-PCR

About twenty cells (containing ˜0.1 ng total RNAs) are broken at 94° C. for 5 minutes and applied to a reverse transcription (RT) reaction (20 μl) on ice, comprising 2 μl of 10×RT&T buffer (400 mM Tris-HCl, pH 8.3 at 25° C., 300 mM KCl, 80 mM MgCl₂, 2M betaine, 100 MM DTT), 1 μM poly(dT)₂₆ primers, dNTPs (0.5 mM each for DATP, dGTP, dCTP and dTTP) and RNase inhibitors (20U). After AMV reverse transcriptase (25U) was added, the reaction was incubated at 42° C. for 1 hour and shifted to 52° C. for another 15 min. The first-strand cDNAs so obtained were collected by a microcon-50 microconcentrater filter and then mixed with terminal transferase (50 U), dGTP (0.5 mM) in 0.5×RT&T buffer.

The reaction was incubated at 37° C. for 15 min, stopped by denaturation at 94° C. for 3 minutes and instantly mixed with 1 μM oligo(dC)₁₀N-T7 promoter primer mixture (7). After briefly centrifuging, Taq/Pwo DNA polymerase mixture (3.5 U) and dNTPs (0.5 mM each for dATP, dCTP and dTTP) were added to form promoter-linked double-stranded cDNAs at 52° C. for 3 minutes and then 68° C. for 10 min. An in-vitro transcription (IVT) reaction (40 ml) was prepared, containing 4 μl of 10×RT&T buffer, above reaction, rNTPs (2 mM each for ATP, GTP, CTP and UTP), 1 μM poly(dT)₂₆ primers, and T7 RNA polymerase (160U). After one hour incubation at 37° C., the mRNA transcripts were directly used for another round of amplification without the tailing reaction.

The RNAPCR protocol is performed following the Example 5 with minor modifications. The quality of amplified RNA library (2 μg) was assessed on a 1% formaldehyde-agarose gel along with other resulting products, such as 20 μg of TRIzol-extracted total RNAs and 2 μg of poly(dT)-column purified mRNAs. The labeling of resulting DNAs can be achieved by the incorporation of labeled deoxynucleotides or analogs during reverse transcription, while that of resulting RNAs is accomplished during the IVT reaction (Shi-Lung Lin et.al. Science (2001)).

### EXAMPLE 12

### Probe Labeling for Microarray Detection

RNAs (2 μg) is converted into double-stranded cDNA by using a modified oligo(dT) primer with a T7 RNA polymerase promoter sequence at the 5′ end, 5′-GGCCAGTGAATTGTAATACGACTCACTCAC TATAGGGAGGCGG-(dT)₂₄-3′, and the Superscript Choice system for cDNA synthesis (GIBCO BRL). Double-stranded cDNA is purified by phenol/chloroform extractions, precipitated with ethanol, and resuspended at a concentration of 0.5 μg/μl in diethyl pyrocarbonate-treated water. Phase-Lock Gel (5′ Prime→3′ Prime, Inc.) is used for all organic extractions to increase recovery. IVT reaction is performed with T7 RNA polymerase and with 1 μg of cDNA, 7.5 mM unlabeled ATP and GTP, 5 mM unlabeled UTP and CTP, and 2 mM biotin-labeled CTP and UTP (biotin-11-CTP, biotin-16-UTP, Enzo Diagnostics). Reactions are carried out for 4 hr at 37° C., and cRNA is purified by RNA affinity resin (RNeasy spin columns, Qiagen). A sample is separated on a 1% agarose gel to check the size range, and then 10 μg of cRNA is fragmented randomly to an average size of 50 bases by heating at 94° C. for 35 minutes in 40 mM Tris-acetate, pH 8.0, 100 mM KOAc/30 mM MgOAc.

The present invention has been described with reference to particular preferred embodiments; however, the scope of this invention is defined by the attached claims and should be constructed to include reasonable equivalents.

Defined in detail, the present invention is an improved polymerase thermocycling reaction method which provides linear amplification of nucleic acid sequences from either cellular RNAs or genomes or both, comprising the steps of:

a) permitting the denaturation of a plurality of nucleic acid templates for the step b) hereafter;

b) obtaining a starting solution by adding to a buffered condition comprising said denatured nucleic acid templates, a primer, a promoter-containing primer, a plurality of deoxynucleotide triphosphates, a plurality of ribonucleotide triphosphates, a sufficient amount of enzyme activities containing reverse transcription, DNA-dependent DNA polymerase and RNA polymerase activities, wherein said buffered condition is sufficient to maintain said enzyme activities in the steps hereafter;

c) contacting said promoter-containing primer with said nucleic acid templates at a predetermined temperature sufficient to form stable annealing interactions, and maintaining said predetermined temperature for sufficient time, whereby a plurality of promoter-containing nucleic acid templates are generated;

d) heating said promoter-containing nucleic acid templates to a temperature sufficient to permit denaturation, and maintaining said temperature for a sufficient time to provide denaturation of said promoter-containing nucleic acid templates without inactivating said enzyme activities containing reverse transcription and DNA-dependent DNA polymerase activities;

e) contacting said primer with said denatured promoter-containing nucleic acid templates at a predetermined temperature sufficient to form stable annealing interactions, and maintaining said predetermined temperature for sufficient time whereby a plurality of promoter-containing double-stranded DNA templates are generated with a desired size sufficient to permit performance of step f) hereafter, wherein said desired size is a plurality of fragment sequences of said nucleic acid templates flanked with said promoter-containing primer in one end and said primer in the other end of the other orientation;

f) permitting transcriptional amplification of said promoter-containing double-stranded DNA templates at a predetermined temperature sufficient to form a plurality of amplified RNA sequences, wherein said amplified RNA sequences are generated by said enzyme activities containing RNA polymerase activities through the promoter region of said promoter-containing double-stranded DNA templates;

g) contacting said amplified RNA sequences with said primer at a predetermined temperature sufficient to form stable annealing interactions, and maintaining said predetermined temperature for sufficient time, whereby a plurality of complementary DNAs are synthesized and a plurality of DNA-RNA hybrid templates are formed; and

h) heating said DNA-RNA hybrid templates to a temperature sufficient to permit denaturation, and maintaining said temperature for a sufficient time to provide denaturation of said DNA-RNA hybrid templates without inactivating said enzyme activities containing reverse transcription and DNA-dependent DNA polymerase activities; so as to provide said denatured nucleic acid templates for the step b) herebefore and hereafter.

Alternatively defined, the present invention is a kit for an improved polymerase thermocycling reaction procedure which provides linear amplification of nucleic acid sequences from either cellular RNAs or genomes or both, comprising the components of:

a) a plurality of nucleic acid templates;

b) a plurality of conditioned buffers;

c) a plurality of primers;

d) a plurality of promoter-containing primers;

e) a plurality of deoxynucleotide triphosphates;

f) a plurality of ribonucleotide triphosphates; and

g) a sufficient amount of enzyme activities containing reverse transcription, DNA-dependent DNA polymerase and RNA polymerase activities.

Of course the present invention is not intended to be restricted to any particular form or arrangement, or any specific embodiment disclosed herein, or any specific use, since the same may be modified in various particulars or relations without departing from the spirit or scope of the claimed invention hereinabove shown and described of which the apparatus shown is intended only for illustration and for disclosure of an operative embodiment and not to shown all of the various forms or modifications in which the present invention might be embodied or operated.

The present invention has been described in considerable detail in order to comply with the patent laws by providing full public disclosure of at least one of its forms. However, such detailed description is not intended in any way to limit the broad features or principles of the present invention, or the scope of patent monopoly to be granted.

What we claim is:

1. An improved polymerase thermocycling reaction method which provides linear amplification of nucleic acid sequences, comprising the steps of:
a. denaturing a plurality of nucleic acid templates;
b. combining said denatured nucleic acid templates with a primer, a promoter-containing primer, a plurality of deoxynucleotide triphosphates, a plurality of ribonucleotide triphosphates, a reverse transcription enzyme, a DNA-dependent DNA polymerase enzyme and an RNA polymerase enzyme;
c. contacting said promoter-containing primer with the nucleic acid templates to generate a plurality of promoter-containing nucleic acid templates;
d. denaturing the promoter-containing nucleic acid templates;
e. contacting the primer with the denatured promoter-containing nucleic acid templates to generate a plurality of promoter-containing double-stranded DNA templates, wherein the double-stranded nucleic acid templates are flanked with said promoter-containing primer in one end and said primer in the other end of the other orientation;
f. transcribing the promoter-containing double-stranded DNA templates to form a plurality of amplified RNA sequences, including the primer region of the promoter-containing double-stranded DNA templates;
g. contacting the amplified RNA sequences with the primer to form a plurality of complementary DNAs and a plurality of DNA-RNA hybrid templates; and
h. denaturing the DNA-RNA hybrid templates.

2. The method as defined in claim 1, further comprising repeating steps (b) through (h) using the denatured DNA-RNA templates at least one time.

3. The method as defined in claim 1, wherein said nucleic acid templates are genomic DNAs.

4. The method as defined in claim 1, wherein said nucleic acid templates are cellular RNA sequences.

5. The method as defined in claim 1, wherein said nucleic acid templates are microbe gene sequences selected from the group consisting of bacteria, virus, fungi and parasite genes.

6. The method as defined in claim 1, further comprising the step of incorporating a plurality of cap-nucleotides into 5′-ends of said amplified RNA sequences for in-vitro translation in the step (f).

7. The method as defmed in claim 6, wherein said cap-nucleotide is selected from the group consisting of P¹-5′-(7-methyl)-guanosine-P³-5′-adenosine-triphosphate, P¹-5′-(7-methyl)-guanosine-P³-5′-guanosine-triphosphate, P¹-5′-(7-methyl)-guanosine-P³-5′-cytidine-triphosphate and P¹-5′-(7-methyl)-guanosine-P³-5′-uridine-triphosphate.

8. The method as defined in claim 1, wherein said reverse transcription enzyme is selected from the group consisting of AMV, M-MuLV, HIV-1 reverse transcriptases and Tth-like thermostable polymerases with reverse transcription activity.

9. The method as defined in claim 8, wherein said reverse transcription is performed at temperature ranged from about 37° C. to about 42° C. for the group consisting of AMV, M-MuLV, HIV-1 reverse transcriptases and ranged from about 65° C. to about 72° C. for Tth-like thermostable polymerases with reverse transcription activity, such as C. therm. polymerase.

10. The method as defined in claim 1, wherein said promoter-containing primer is coupled to a promoter site in the 5′-end for the initiation of RNA polymerase activity selected from the group consisting of T3, T7, SP6 and M13 RNA polymerases.

11. The method as defined in claim 10, wherein said transcription is performed at temperature ranged about 37° C.

12. The method as defined in claim 1, wherein said denaturation is accomplished by heating.

13. The method as defined in claim 12, wherein the heating is at a temperature ranged about 94° C.

14. The method as defmed in claim 1, wherein said DNA-dependent DNA polymerase enzyme is selected from the group consisting of E. coli DNA polymerase 1, Klenow fragment of E. coli DNA polymerase 1, T4 DNA polymerase, Taq DNA polymerase, Pwo DNA polymerase, Pfu DNA polymerase and Tth-like thermostable polymerases with reverse transcription activity.

15. The method as defined in claim 14, wherein said DNA polymerase enzyme is a Tth-like thermostable polymerase.

16. The method as defined in claim 15, wherein said Tth-like thermostable polymerase is a C. therm. polymerase.

17. The method as defined in claim 14, wherein said DNA double-stranding activity is performed at a temperature range of about 370° C. for the group consisting of E. coli DNA polymerase 1, Klenow fragment of E. coli DNA polymerase 1 and T4 DNA polymerase, and about 70° C. for Taq DNA polymerase, Pwo DNA polymerase, Pfu DNA polymerase and Tth-like thermostable polymerases.

18. The method as defined in claim 1, wherein said buffered condition is provided by a chemical reagent containing Tris-HCl, NaCl/KCl, MgCl₂, (NH₄)₂SO₄/betaine, dithioerythritol/dithiothreitol and/or spermidine.

19. The method of claim 1, wherein said primer and promoter-containing primer is of either gene-specific sequences or random sequences.

20. The method of claim 19, wherein said random sequences comprise a random hexamer.

21. A kit providing an improved polymerase thermocycling reaction procedure which accomplishes the linear amplification of nucleic acid sequences from either cellular RNAs or genomes or both, comprising the components of:
a. a plurality of nucleic acid templates;
b. a plurality of primers;
c. a plurality of promoter-containing primers;
d. a plurality of deoxynucleotide triphosphates;
e. a plurality of ribonucleotide triphosphates;
f. a sufficient amount of enzyme activities containing reverse transcription, DNA-dependent DNA polymerase and RNA polymerase activities; and
g. a plurality of conditioned buffers, wherein said buffers are optimized to provide functional conditions for said enzyme activities in the component (f).

22. The kit as defined in claim 21, further comprising a plurality of cap-nucleotides, wherein said cap-nucleotides can be added to 5′-ends of said amplified RNA sequences for in vitro translation in the step (f) of claim 1.

23. The kit as defined in claim 22, wherein said cap-nucleotide is selected from the group consisting of P¹-5′-(7-methyl)-guanosine-P³-5′-adenosine-triphosphate, P¹-5′-(7-methyl)-guanosine-P³-5′-guanosine-triphosphate, P¹-5′-(7-methyl)-guanosine-P³-5′-cytidine-triphosphate and P¹-5′-(7-methyl)-guanosine-P³-5′-uridine-triphosphate.

24. The kit as defined in claim 21, wherein said nucleic acid templates are genomic DNAs.

25. The kit as defined in claim 21, wherein said nucleic acid templates are cellular RNA sequences.

26. The kit as defined in claim 21, wherein said nucleic acid templates are microbe gene sequences selected from the group consisting of bacteria, virus, fungi and parasite genes.

27. The kit as defined in claim 21, wherein said enzyme activities contain a plurality of mixed polymerase activities from DNA-dependent DNA, DNA-dependent RNA polymerases, and RNA-dependent DNA polymerases.

28. The kit as defined in claim 27, wherein said DNA-dependent DNA polymerase is selected from the group consisting of E. coli DNA polymerase 1, Klenow fragment of E. coli DNA polymerase 1, T4 DNA polymerase, Taq DNA polymerase, Pwo DNA polymerase, Pfu DNA polymerase and Tth-like thermostable polymerases with reverse transcription activity.

29. The kit as defined in claim 27, wherein said DNA-dependent RNA polymerase is selected from the group consisting of T3, T7, SP6 and M13 RNA polymerase.

30. The kit as defined in claim 27, wherein said RNA-dependent DNA polymerase is selected from the group consisting of AMV, M-MuLV, HIV-1 reverse transcriptases and Tth-like thermostable polymerases with reverse transcription activity.

31. The kit as defined in claim 21, wherein said deoxynucleotide triphosphates contain deoxyguanylate triphosphates, deoxycytidylate triphosphates, deoxyadenylate triphosphates, deoxythymidylate triphosphates, deoxyuridylate triphosphates and the deoxynucleotide analogs thereof.

32. The kit as defined in claim 21, wherein said ribonucleotide triphosphates contain guanylate triphosphates, cytidylate triphosphates, adenylate triphosphates, uridylate triphosphates and the ribonucleotide analogs thereof.

33. The kit as defined in claim 21, wherein said conditioned buffer is a chemical reagent containing Tris-HCl, NaCl/KCl, MgCl₂, (NH₄)₂SO₄/betaine, dithioerythritol/dithiothreitol and/or spermidine.

34. A method of producing a mRNA expression profile of a cell using the method of claim 1 to generate multiple copies of said mRNA.

35. The method of claim 34, wherein the generation of multiple copies of said mRNA is for the purposes of generating a protein expression profile of a cell.

36. The method of claim 1 used for the purposes of producing an amplified genomic DNA sequence or sequences for a specific gene or genes or for the whole genome genomic DNAs.

37. The method of claim 36, used for the purposes of producing amplified DNA sequence or sequences to be utilized in a genotyping scheme.

38. The method of claim 37, wherein said genotyping scheme is a method for screening and/or analyzing differences between compositions of different expression profiles.

39. The method of claim 38, wherein said compositions are prepared by using the method of claim 1.

40. The method of claim 39, wherein said compositions are of either a single-stranded or a double-stranded nucleic acid.

41. The method of claim 40, wherein said nucleic acid is of either DNA, RNA or a hybrid of DNA and RNA.

42. A method for determining aberrant protein production of cells in a diseased state, comprising the steps of:
(a) generating an expression profile, according to the method of claim 34 or 35, of cells in both normal and diseased states;
(b) comparing the expression profile of the cells in the normal and diseased states;
(c) determining the differences in mRNA composition of the cell(s) in the diseased state;
(d) isolating said mRNA sequences of said cell(s) in the diseased state that differ from mRNA in cell(s) in non-diseased state;
(e) using the method according to claim 1, to amplify said isolated mRNA; and
(f) determining aberrant protein function of the protein coded for by said isolated mRNA.

43. The method of claim 42, wherein steps (a) to (e) are practiced for the purpose of determining the protein sequence for which said mRNA codes.

44. The method of claim 42, wherein steps (a) to (e) are practiced for the purpose of determining the nucleotide sequence encoding said mRNA.

45. A method for treating a cell in a diseased state caused by aberrant protein production, comprising the steps of:
(a) determining protein expression of a cell in a diseased state;
(b) determining the mRNA sequence for said aberrant proteins;
(c) synthesizing an anti-sense sequence to said mRNA;
(d) using the method of claim 1 to amplify said anti-sense mRNA sequences; and
(e) delivering a pharmaceutically effective dosage of a composition comprising said anti-sense mRNA and a compatible lipid based biological carrier.

46. A method for predicting the efficacy of a proposed drug targeted against an aberrant protein, comprising the steps of:
(a) determining aberrant protein production of cells in a diseased state according to claim 42;
(b) using the method of claim 1 to amplify the aberrant protein; and
(c) using recombinant techniques to determine the effect of said proposed drug on the aberrant protein.

47. A method for the differential screening of tissue-specific gene expression at a cellular level, comprising the steps of:
a. generating a gene expression profile, according to the method of claim 1, of cells which are dissected from a homogeneous cell region of a tissue sample by a single-cell isolation device selected from the group including tissue-shredder columns, micromanipulators and laser-capture devices;
b. comparing the gene expression profile of the cells to another gene expression profile by microarray and/or subtractive hybridizations; and
c. selecting the differently expressed genes from the compared gene expression profiles; so as to provide a differentially expressed gene profile of the tissue cells.

48. A method for preparing labeled RNA/DNA probes for a gene chip technology, comprising the steps of:
a. generating a mRNA library, according to the method of claim 1, of cells of interest;
b. combining said mRNA library with a primer, a promoter-containing primer, a kind of labeled deoxynucleotide or ribonucleotide triphosphates, a plurality of unlabeled deoxynucleotide and ribonucleotide triphosphates, a reverse transcription enzyme, a DNA-dependent DNA polymerase enzyme and an RNA polymerase enzyme;
c. contacting said promoter-containing primer with the RNA templates of said mRNA library to generate a plurality of promoter-containing nucleic acid templates;
d. denaturing the promoter-containing nucleic acid templates;
e. contacting the primer with the denatured promoter-containing nucleic acid templates to generate a plurality of promoter-containing double-stranded DNA templates, wherein the double-stranded nucleic acid templates are flanked with said promoter-containing primer in one end and said primer in the other end of the other orientation;
f. transcribing the promoter-containing double-stranded DNA templates to form a plurality of labeled RNA probes;
g. reverse-transcribing said RNA probes to form a plurality of labeled DNA probes; and
h. contacting said labeled RNA or DNA probes with a gene chip or microarray; so as to provide an expression profile of the interested cells.

49. The method as defined in claim 48, wherein said labeled nucleotide triphosphates is labeled by a chemical selected from the group consisting essentially of biotin, avidin, digoxigenin fluorescein, Cy3, Cy5 and radioactive isotopes.

50. A method for cloning full-length sequences of unknown gene transcripts, comprising the steps of:
a. denaturing a plurality of nucleic acid templates containing a gene or its transcript which is desired to be cloned;
b. combining said denatured nucleic acid templates with a primer complementary to the 3′-end of said desired gene, a promoter-containing primer homologous to the 5′-end of said desired gene, a plurality of deoxynucleotide triphosphates, a plurality of ribonucleotide triphosphates, a reverse transcription enzyme, a DNA-dependent DNA polymerase enzyme and an RNA polymerase enzyme;
c. contacting said primer with the nucleic acid templates to generate a plurality of primer-containing nucleic acid templates of said desired gene;
d. denaturing the primer-containing nucleic acid templates;
e. contacting the promoter-containing primer with the denatured primer-containing nucleic acid templates to generate a plurality of promoter-containing double-stranded DNA templates, wherein the double-stranded nucleic acid templates are flanked with said promoter-containing primer in the 5′-end and said primer in the 3′-end of the other orientation; and
f. transcribing the promoter-containing double-stranded DNA templates to form a plurality of amplified RNA sequences of said desired gene; and so as to provide a clone of the desired gene in the form of RNA and promoter-containing double-stranded DNA templates.

51. A method for determining the efficacy of a drug regiment against a gene or its cDNAs, comprising the steps of:
a. generating an expression profile, according to the method of claim 34 or 35, of cells which are treated by a drug;
b. generating an expression profile, according to the method of claim 34 or 35, of cells which are not treated by said drug;
c. comparing the gene expression profile of the drug-treated cells in step (a) to the gene expression profile of the untreated cells in the step (b) by microarray and/or subtractive hybridizations; and
d. selecting the differently expressed genes from the compared gene expression profiles; so as to provide the gene and/or its cDNAs which are sensitive to the treatment of said drug.


SEQUENCE LISTING (12 sequences)

SEQ ID NO: 1 - DNA, 57 bp, artificial sequence
  Oligo(dT)-T7 promoter primer
ccagtgaattgtaatacgactcactatagggaatttttttttttttttttttttttt

SEQ ID NO: 2 - DNA, 18 bp, artificial sequence
  poly(C) primer
cccccccccccccccccc

SEQ ID NO: 3 - DNA, 41 bp, artificial sequence
  Oligo(dC)-SP6 promoter primer
ccatatggcatttaggtgacactatagaagccccccccccc

SEQ ID NO: 4 - DNA, 26 bp, artificial sequence
  poly(dT) primer
tttttttttttttttttttttttttt

SEQ ID NO: 5 - DNA, 43 bp, artificial sequence
  oligo(dC)-T7 promoter primer
ccagtgaattgtaatacgactcactatagggaacccccccccc

SEQ ID NO: 6 - DNA, 24 bp, artificial sequence
  Oligo(dT) primer for RNA polymerase thermocycling procedure
tttttttttttttttttttttttt

SEQ ID NO: 7 - DNA, 42 bp, artificial sequence
  Oligo(dC) -N-T7 promoter primer for RNA polymerase thermocycling procedure
ccagtgaattgtaatacgactcactataggcccccccccccn

SEQ ID NO: 8 - DNA, 10 bp, artificial sequence
  primer for RNA polymerase thermocycling procedure
nnnnnnnnnn

SEQ ID NO: 9 - DNA, 41 bp, artificial sequence
  SP6 promoter-containing degenerate sequence primer for RNA polymerase thermocycling procedure
ccatatggcatttaggtgacactatagaagcnnnnnnnnnn

SEQ ID NO: 10 - DNA, 20 bp, artificial sequence
  antisense bcl-2 primer
cttcttcaggccagggaggc

SEQ ID NO: 11 - DNA, 49 bp, artificial sequence
  T7 promoter-containing sense-bcl2 primers
ccagtgaattgtaatacgactcactataggccgttacttttcctctggg

SEQ ID NO: 12 - DNA, 67 bp, artificial sequence
  oligo(dT) primer with a T7 RNA polymerase promoter sequence at the 5′ end.
ggccagtgaattgtaatacgactcactcactatagggaggcggtttttttttttttttttttttttt